(19) United States
(12) Patent Application Publication
(10) Pub. No.: US 2023/0272020 A1
(43) Pub. Date: Aug. 31, 2023

(54) MODIFIED SEMAPHORIN 3A, COMPOSITIONS COMPRISING THE SAME AND USES THEREOF

(71) Applicants: MEDICAL RESEARCH & DEVELOPMENT FUND FOR HEALTH SERVICES BNAI ZION MEDICAL CENTER, Haifa (IL); TECHNION RESEARCH & DEVELOPMENT FOUNDATION LIMITED, Haifa (IL); THE CHANCELLOR, MASTERS AND SCHOLARS OF THE UNIVERSITY OF OXFORD, Oxford (GB)

(72) Inventors: Zahava VADASZ, Haifa (IL); Elias TOUBI, Haifa (IL); Nasren EIZA, Haifa (IL); Adi SABAG, Binyamina-Giv'at Ada (IL); Gera NEUFELD, Haifa (IL); Ofra KESSLER, Tel Aviv (IL); E. Yvonne JONES, Oxford (GB)

(21) Appl. No.: 17/928,685

(22) PCT Filed: Jun. 3, 2021
(86) PCT No.: PCT/IL2021/050660

§ 371 Date: Nov. 30, 2022

Related U.S. Application Data
(60) Provisional application No. 63/034,476, filed on Jun. 4, 2020.

(51) Int. Cl. C07K 14/47; A61P 29/00; A61P 37/00; C12N 15/86

(52) U.S. Cl. CPC C07K 14/47; A61P 29/00; A61P 37/00; C12N 15/86

(57) ABSTRACT

Provided herein are modified forms of Semaphorin 3A (Sema3A) polypeptide having one or more amino acid substitutions and/or deletions compared to a wild type Sema3A protein. Further provided are nucleic acid molecules encoding the modified Sema3A polypeptide, compositions including the same and uses thereof in treating various immune-related conditions.

### FIELD OF THE INVENTION

The present invention relates to modified forms of Semaphorin 3A (Sema3A) polypeptide having amino acid(s) substitution and/or deletion compared to a wild type Sema3A protein. The invention further relates to compositions including the modified Sema3A and uses thereof for treating various immune-related conditions.

### BACKGROUND OF THE INVENTION

Semaphorins are a family of membrane bound and soluble proteins classified into eight sub-classes based on their structural domains. Semaphorins were found to regulate axon guidance, organogenesis, angiogenesis, lymphangiogenesis and immune responses and to modulate tumor progression. The Semaphorins are divided into several subfamilies.

The seven class-3 Semaphorins (Semaphorin 3s), designated by the letters A-G, are the only vertebrate secreted Semaphorins. Neuropilins (Nrps) and the type A/D family Plexins (Plexin-A1, -A2, A3, A4 and Plexin-D1) act as receptors for class-3 Semaphorins. Each Semaphorin 3 family member shows distinct binding preference for Nrps. Each Sema3-Nrp complex associates with specific plexins to mediate downstream signaling, including transducing signals that induce the collapse of the actin cytoskeleton of target cells. Most membrane-bound vertebrate Semaphorins directly bind plexins, while the class-3 Semaphorins, with the exception of sema3E and sema3C, require Neuropilins as obligate co-receptors.

Semaphorin 3A (Sema3A), a class-3 secreted member of the Semaphorin family, has been established as an axonal guidance factor during development. Sema3A has also been shown to be expressed by activated T cells and inhibit T cell proliferation and cytokine secretion. Additionally, Neuropilin-1 expression on regulatory T cells has been shown to enhance interactions with immature dendritic cells (DCs) during antigen recognition, resulting in higher sensitivity to limiting amounts of antigen. In addition to its role as an axon guidance factor, Sema3A functions as an inhibitor of angiogenesis and as a blood vessels permeabilizing agent, functions mediated through the neuropilin-1 receptor. Sema3A also functions as an inhibitor of tumor progression in a variety of solid tumors as well as in hematological malignancies such as multiple myeloma.

Sema3A was also characterized as a modulator of immune responses. It inhibits primary T-cell proliferation and pro-inflammatory cytokines production under anti-CD3 plus anti-CD28 stimulating conditions and inhibits the migration of thymocytes. Sema3A production by bone marrow derived mesenchymal stem cells seems to mediate at least part of their immune suppressive effects. In addition, sema3A was suggested to have beneficial effects in a variety of auto-immune diseases. For example, it was found that sema3A reduced kidney failure in NZB/W mouse model of lupus nephritis and reduced the severity of asthma in mouse models of asthma and allergic rhinitis. Such beneficiary effects were likely due in part to sema3A stimulation of FoxP3 and IL-10 expression in Treg cells and the significant reduction in TLR-9 expression in B cells. It was found that the concentration of Sema3A is strongly reduced in the sera of patients afflicted with immune-mediated (e.g. Familial Mediterranean fever (FMF)) and auto-immune diseases such as systemic lupus erythematosus and systemic sclerosis. Furthermore, it was found that systemic administration of recombinant Sema3A inhibits the development of kidney failure in the NZB/W mouse model of lupus nephritis, and alleviates asthma in an asthma model, It was further found that Sema3A promotes the expression of immune suppressive cytokines such as IL-10 from regulatory T cells (Treg) and the expansion of a subpopulation of regulatory B cells (Breg) that highly express IL10, suggesting that Sema3A is a master regulator that inhibits immune responses, at least in part, by the regulation of the expression of inhibitory cytokines.

Thus, for example, U.S. Pat. No. 10,105,413 relates to Semaphorin 3A and use thereof in treatment and prognosis of Systemic Lupus Erythematosus (SLE). U.S. Pat. No. 10,568,932 is related to Semaphorin 3A for treatment and assessment of severity of asthma. International publication No. 2016/128966 relates to Semaphorin 3A for treatment and assessment of severity of Inflammatory Bowel Disease (IBD).

International application WO 2016135130 relates to non-natural Semaphorins 3 and their medical use and discloses various mutated Semaphorin 3 molecules and methods of using them in the treatment of disease, in particular in the medical intervention of angiogenic diseases, tumors and/or cancer.

Nevertheless, there is a need in the art for modified forms of Sema3A that exhibit improved properties, compared to unmodified Sema3A molecules, and which can be used for safe, efficient and cost effective treatment of various immune-related conditions.

### SUMMARY OF THE INVENTION

According to some embodiments, there is provided an advantageous modified Semaphorin 3A polypeptide, which includes one or more point mutations and/or truncations, compared to a wild-type (non-modified) Semaphorin 3A. According to some embodiments, the novel, non-naturally occurring, modified Sema3A disclosed herein is advantageous, as it is stable, easy to produce, and exhibit a desired biological activity, as further detailed herein. Further provided are nucleic acids encoding for the modified Sema3A polypeptide, methods for the preparation of the modified Sema3A, compositions comprising the same and uses thereof in treating various medical conditions, in particular, immune-related conditions.

According to some embodiments, the advantageous modified/non-naturally occurring/genetically modified/mutated Semaphorin 3A polypeptide includes at least one point mutation and/or deletion (truncation) of a stretch of amino acids, compared to a WT, unmodified, naturally occurring Sema3A.

According to some embodiments, the modified Sema3A (also referred to herein as “T-sema3A”) includes one amino acid substitution and a C-terminal deletion (of at least 100 amino acids), as compared to a WT Sema3A.

In some embodiments, the modified Sema3A includes an amino acid substitution in position 257 of the human amino acid sequence of wild type Sema3A (represented by amino acid sequence denoted by SEQ ID NO: 1), whereby the amino acid Serine (Ser) in the WT sequence is replaced by amino acid Cysteine (Cys). Thus, the modified Sema3A includes a S257C sequence substitution. In some exemplary embodiments, the modified Sema3A further includes a deletion/truncation of 254 amino acids from the C-terminus of the WT Sema3A. That is, the modified Sema3A is truncated at amino acid 516 of the WT Sema3A. In some exemplary embodiments, the modified Sema3A polypeptide comprises an amino acid sequence as denoted by SEQ ID NO: 3.

According to further embodiments, the modified Sema3A may further include one or more additional tag sequences at the N-terminal and/or C-terminal thereof In some embodiments, the Tag sequence may be used for marking/identification and/or purification of the modified Sema3A. In some embodiments, the tag sequence may be selected from His tag (i.e., including a stretch of Histidine amino acids, for example, 8 Histidine amino acids), FLAG-tag, Myc-tag, and the like. The tag sequences may be placed in-frame at the N-terminal of the modified proteins and/or on the C-terminal of the modified protein. In some exemplary embodiments, the modified Sema3A protein may include a stretch of 8 Histidine (8-His-Tag) at the C-terminus of the polypeptide.

According to some embodiments, as mentioned above, WT Sema3A binds to the neuropilin-1 receptor (nrp1) which subsequently associates with type-A plexin receptors that function as the signal transducing elements in the functional sema3A receptor. Classically, signaling via these receptor complexes induces the collapse of the cytoskeleton in target cells. Surprisingly, the inventors of the present application have revealed that CD72 receptor also functions as a sema3A receptor (in addition to the known neuropilin-1 which was considered to be the sole sema3A binding receptor), and that CD72 mediated signal transduction can control anti-inflammatory gene expression in primary B-lymphoblastoid cells lacking neuropilin receptors. Thus, as disclosed herein, the anti-immune effects of sema3A may be mediated, at least in part, by the CD72 receptor. Accordingly, without wishing to be bound to any theory or mechanism, the advantageous, non-naturally occurring modified Sema3A exhibits a differential activation as compared to a WT Sema3A. In other words, the modified Sema3A protein, having a truncation at the C-terminal region of the protein, will not be able to activate neuropilin-1 mediated signal transduction, but does retain its ability to activate CD72 mediated signal transduction. Thus, the advantageous modified Sema3A protein disclosed herein may retain its anti-immune properties, mediated via CD72 binding, yet be devoid of undesired side effects which in the wild type sema3A are mediated via the neuropilin-1 receptor. Further, since the Sema3A is active as a homodimer, in order to allow the modified Sema3A to retain dimerization capabilities (which are found in the WT protein in the C-terminal region), the S257C point mutation mentioned above was introduced.

According to some embodiments, as further exemplified herein, the advantageous modified Sema3A retains the immune beneficiary properties of wild type Sema3A while and because it interacts with only a subset the sema3A receptors, displays fewer side effects, as compared with wild type sema3A. Further, as exemplified herein, the modified Sema3A was found to be at least as effective as wild type sema3A in increasing T regulatory cells function. In further embodiments, as disclosed herein, the modified sema3A is capable of reducing activity and metabolism of activated T-cells. According to some embodiments, T-Sema3A can affect (decrease) the glycolytic rate of activated T-cells, i.e., down regulate aerobic glycolysis in such activated immune cells.

Accordingly, in some embodiments, the modified-sema3A can therefore be used for the successful treatment of various immune-mediated conditions, such as, auto-immune diseases (such as, for example, Systemic Lupus Erythematosus (SLE), Rheumatoid Arthritis, inflammatory bowel disease (IBD), Uveitis, Psoriasis), allergic conditions (such as, bronchial asthma, allergic conjunctivitis, allergic rhinitis and atopic dermatitis), conditions related to over activation of the immune system (such as, for example, sepsis, cytokine storm-due to infectious diseases and/or CAR-T treatment, graft-versus host disease (GVHD), inflammatory diseases (such as, Chronic Obstructive Pulmonary Disease (COPD), Familial Mediterranean fever (FMF)). In some exemplary embodiments, the immune-mediated condition may include, for example, Systemic Lupus Erythematosus (SLE), asthma, IBD, and the like.

According to some embodiments, there is thus provided a novel, non-naturally occurring modified sema3A (T-sema3A) that is unable to signal via neuropilins yet capable of displaying anti-inflammatory effects at least as good as, if not better, compared to wild type sema3A in various assays. The disclosed T-sema3A is advantageous as it is smaller in size, compared to the wild type sema3A, and may therefore be more diffusible and less difficult to produce in large quantities. In addition, it may be safer and more potent for use in treating various immune-mediated disorders. Wild type sema3A has beneficial effects in several autoimmune diseases. However, it also affects additional biological processes such as angiogenesis and axon guidance as a result of its binding to receptors of the neuropilins family. Thus, treatment with wild type sema3A may be accompanied by diverse side effects resulting from the activation of neuropilins mediated signaling in various body compartments. Thus, without wishing to be bound to any theory or mechanism, the disclosed T-sema3A, which retains the immune beneficial effects of wild type sema3A, but un-able to activate he undesired neuropilin mediated signal transduction, may consequently exhibit fewer side effects. Thus, according to some embodiments, the herein disclosed modified Sema3A surprisingly exhibit better in vivo and/or in vitro properties as compared to naturally occurring Sema3A (WT-Sema3A). In some embodiments, the modified Sema3A disclosed herein exhibit improved therapeutic activity of immune-related conditions, as compared to a WT Sema3A. In some embodiments, the modified Sema3A exhibit one or more improved properties as compared to a WT Sema3A, the properties may include: pharmacologic effects, pharmacokinetic, stability, half-life, delivery, efficiency, cellular targets, side effects, and the like, or any combination thereof.

According to some embodiments, in view of the S257C substitution introduced in the T-Sema3A, the modified Sema3A can function as a dimer, whereby two monomeric T-Sema3A can form a dimer, via sulfide bonds, between the respective Cysteine residues introduced into the sequence. Therefore, the modified Sema3A proteins disclosed herein can preferably and advantageously be in the form of the dimer. In some embodiments, the thus formed dimer is a homo-dimer. The term “homo-dimer” indicates that two identical T-Sema3A monomers are in the form of a dimer.

In some embodiments, the two monomers of the dimer can be comprised in one fusion protein. In some embodiments, the two modified Sema3A monomers of the dimer may be encoded by a single nucleic acid molecule. In some embodiments, the two monomers of the dimer can be formed independently in a tube or a cell, and form a dimer in-vitro or in-vivo, for example, after being produced or placed under physiological conditions.

According to some embodiments, as exemplified herein, it was surprisingly found that the replacement of the S257C and the truncation of the C-terminal region (which includes the native binding region of the Nrp1 receptors), results in binding to CD72 receptor, independently of Nrp1 binding. As further exemplified herein the modified Sema3A exhibit activation of regulatory T-cells. For example, the modified Sema3A can bind to cellular CD72 receptor. For example, the modified Sema3A can active CD4+ regulatory T-cells and induce IL-10 secretion. For example, the modified Sema3A does not induce cell-contraction.

According to some embodiments, provided are methods and compositions for treatment of immune-related condition, said methods comprising administration of a pharmaceutical composition comprising the modified Semaphorin 3A to a subject in need thereof In some embodiments, the immune-related condition is selected from Asthma, IBD and systemic Lupus Eryhtmus (SLE).

According to some embodiments, there is provided a modified Semaphorin 3A polypeptide, the modified Semaphorin 3A polypeptide includes an amino acid substitution/replacement at a position corresponding to position 257 in a wild type Semaphorin 3A protein having an amino acid sequence as denoted by SEQ ID NO: 1, wherein the replacement is with Cysteine (C); and a deletion of at least 100 amino acids of the C-terminal region of the corresponding wild type Semaphorin 3A.

According to some embodiments, the amino acid substitution is S257C and the C-terminal deletion is of amino acids 517-771 of the corresponding wild type Semaphorin 3A.

According to some embodiments, the modified Semaphorin 3A and the wild type Semaphorin 3A are of human origin.

According to some embodiments, the polypeptide may further include a Tag sequence at the N-terminus and/or the C-terminus thereof.

According to some embodiments, tag sequence is positioned in frame at the C-terminal region of the polypeptide. According to some embodiments, the Tag sequence is selected from: His-Tag, Myc-Tag and FLAG-tag.

According to some embodiments, the Tag sequence may include a stretch of 6 or more consecutive Histidine residues.

According to some embodiments, the modified Semaphorin 3A polypeptide has an amino acid sequence as denoted by SEQ ID NO: 3. According to some embodiments, the modified Semaphorin 3A polypeptide has an amino acid sequence as denoted by SEQ ID NO: 5.

According to some embodiments, the modified Semaphorin 3A polypeptide is configured to or is capable of forming a homo-dimer with a modified Semaphorin 3A polypeptide via S-S bonds formed between Cysteine 257 in each of the modified polypeptides.

According to some embodiments, the modified Semaphorin 3A polypeptide is capable of binding CD72 receptor.

According to some embodiments, the modified Semaphorin 3A polypeptide un-capable of binding to Nrp1.

According to some embodiments, the modified Semaphorin 3A polypeptide is unable to induce cell contraction.

According to some embodiments, the modified Semaphorin 3A polypeptide s capable of inducing/changing/affecting expression of one or more anti-inflammatory cytokines.

According to some embodiments, the modified Semaphorin 3A polypeptide is capable of inducing expression of IL-10 in CD4+ regulatory T-cells.

According to some embodiments, there is provided a composition comprising the modified Semaphorin 3A polypeptide disclosed herein.

According to some embodiments, the modified Semaphorin 3A polypeptide disclosed herein, or the composition comprising the same may be used for treating an immune-related condition in a subject in need thereof.

According to some embodiments, the immune related condition is selected from Asthma, SLE and IBD.

According to some embodiments, there is provided a nucleic acid molecule (polynucleotide) encoding the modified Semaphorin 3A disclosed herein.

According to some embodiments, the nucleic acid molecule encoding the modified Semaphorin 3A has a nucleotide sequence as denoted by any one of SEQ ID NO: 4 and SEQ ID NO: 6.

According to some embodiments, there is provided a vector including the nucleic acid molecule encoding for the modified Semaphorin 3A. In some embodiments, the vector is an expression vector, further including one or more regulatory sequences.

According to some embodiments, the nucleic acid molecule encoding the modified Semaphorin 3A or the vector including the nucleic acid may be used for treating an immune-related condition in a subject in need thereof.

According to some embodiments, there is provided a method of treating an immune related condition in a subject in need thereof, the method includes administering to the subject in need thereof a therapeutically effective amount of the modified Sema3A polypeptide disclosed herein, or a composition including the same.

According to some embodiments, there is provided a method of treating an immune related disorder in a subject in need thereof, the method includes administering to the subject in need thereof a therapeutically amount of nucleic acid molecule encoding the modified Semaphorin 3A or the vector including the same.

According to some embodiments, there is provided a host cell harboring the nucleic acid encoding the modified Sema3A.

According to some embodiments, there is provided a host cell transformed or transfected with the vector including the nucleic acid molecule encoding the modified Sema3A.

According to further embodiments, there is provide a host cell which includes or expresses the modified Sema3A polypeptide disclosed herein.

According to some embodiments, there is provided a method of producing the modified Sema3A polypeptide, the method includes the steps of: (i) culturing the host cells under conditions such that the polypeptide comprising the modified Sema3A is expressed; and (ii) optionally recovering the modified Sema3A from the host cells or from the culture medium.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-C—Presents amino acid and nucleotide sequences of human WT-Sema3A and modified Sema3A. FIG. 1A Shows the 771 amino acid sequence of the human WT-Sema3A (SEQ ID NO: 1), with the C-terminal region (amino acids 517-771) marked (gray background); FIG. 1B shows the amino acid sequence of a modified Sema3A (T-sema3A), which includes a S257C substitution (marked by Capital C), and a C-terminal truncation at amino acid R516. The modified Sema3A sequence presented in FIG. 1B further includes an in-frame 8×-His tag (HHHHHHHH (SEQ ID NO: 7 (marked)) at the C-terminal end of the modified protein. The amino acid sequence presented in FIG. 1B corresponds to SEQ ID NO: 5; FIG. 1C presents the nucleic acid sequence of the cDNA encoding for a modified His-tagged Sema3A. The cDNA sequences presented in FIG. 1C corresponds to SEQ ID NO: 6, and includes a codon modification (bases 769-771), whereby the codon encoding for a Serine residue (in the WT Sema3A protein, SEQ ID NO: 2), was changed to a tgt codon encoding cysteine at bases 769-771. In addition, a cDNA sequence encoding 8 histidine residues (caccatcaccatcaccatcaccatcaccat (SEQ ID NO: 8), highlighted) was fused in frame at nucleotide 1548, followed by a stop codon (tga);

FIG. 2—shows a vector map of the NSPI-CMV-MCS-myc-His lentiviral expression vector which harbors a coding sequence of the modified Sema3A, according to some embodiments;

FIGS. 3A-D—Sema3A binds to the CD72 receptor: FIG. 3A — shows pictogram of Western Blot analysis of cell extracts probed with antibodies directed against neuropilin-1 and/or CD72. The cells include Parental U87MG cells (par), cells in which the gene expressing neuropilin-1 was knocked out using CRISPR/Cas9 (U87MG-ΔNrp1), and cells in which the gene expressing neuropilin-1 was knocked out and that were further infected with empty lentiviruses or lentiviruses directing expression of CD72 (U87MG-ΔNrp1+CD72) to which a V5 epitope tag was fused in frame upstream of the stop codon; FIG. 3B—shows pictograms of the cells to which Sema3A-AP was bound to. The cells were consequently washed and bound sema3A-AP was detected using BICP/NBT; FIG. 3C—presents line graphs showing the effect of increasing concentrations of purified Sema3A-AP that were bound for 30 minutes at room temperature to the three cell types. Following binding, the cells were washed and the amount of bound Sema3A-AP per microscopic field was assessed using an alkaline phosphatase colorimetric assay; FIG. 3D—Sema3A-AP (5 μg/ml) was bound to the three cell types in the presence of increasing concentrations of sema4D. The amount of bound sema3A-AP/microscopic field was then determined and presented in the line graphs of FIG. 3D;

FIGS. 4A-C—Sema3A transduces signals using CD72. FIG. 4A—shows pictograms of Western Blot analysis in which a primary B-lymphoblastoid cell line (BLCL) was infected with lentiviruses directing expression of CD72. The BLCL cells and the BLCL cells expressing CD72 were probed with antibodies directed against neuropilin-1 (Nrp1) and CD72. Parental BLCL cell do not express either of these receptors. FIG. 4B and FIG. 4C—BLCL cells and BLCL cells expressing CD72 (BLCL+CD72) were stimulated with Sema3A. The phosphorylation state (p) of STAT-4 (FIG. 4B) and P38 (FIG. 4C) were than determined. Shown in the figures are Western blots probed with antibodies directed against the total (t) proteins and against specific phosphorylation (p) sites in Stat-4 protein (FIG. 4B) and P38 (FIG. 4C). Also shown are bar graphs representing quantification of the Western blot results (i.e., the ratio between phosphorylated (p) and total (t) STAT-4 and P-38);

FIG. 5A-B—Modified Sema3A (T-Sema3A) transduces signals using the CD72 receptor but is unable to induce endothelial cell contraction mediated by the neuropilin-1 receptor: FIG. 5A shows pictograms of Human umbilical vein derived endothelial cells (HUVEC) that were stimulated with conditioned medium from control HEK293 cells (Control), or with conditioned medium containing similar concentrations of WT Sema3A or T-sema3A derived from HEK293 cells expressing either recombinant WT Sema3A or T-sema3A. Cells were photographed 30 minutes after addition of the conditioned media; FIG. 5B shows bar graphs of the percentage of T-cells expressing IL-10 as determined using FACS analysis. CD4⁺ T-cells were stimulated with the indicated concentrations of purified WT Sema3A or T-Sema3A. *=P<0.05;

FIG. 6 shows line graphs of glycolysis stress test of activated T-cells in the presence or absence of T-Sema3A. Purified CD4+ T cells were activated with anti-CD3 and anti-CD28 for 24 hours at 37° C. The activated cells were treated with 5 μg/ml T-Sema3A or PBS (as a control) and incubated for 24 hours at 37° C. Cells were harvested and transferred to medium without glucose for 2 hours. Thereafter, Glucose, Oligomycin and 2-deoxy-glucose (2-DG), were added to the cells at the indicated time points. The real-time (in live) ECAR (extracellular acidification rate) measurements were performed using Agilent Seahorse XF Analyzers (Seahorse Real-Time Cell Metabolic Analysis assay (Agilent)).

### DETAILED DESCRIPTION OF THE INVENTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

As referred to herein, the terms “polynucleotide molecules”, “oligonucleotide”, “polynucleotide”, “nucleic acid” and “nucleotide” sequences may interchangeably be used. The terms are directed to polymers of deoxyribonucleotides (DNA), ribonucleotides (RNA), and modified forms thereof in the form of a separate fragment or as a component of a larger construct, linear or branched, single stranded (ss), double stranded (ds), triple stranded (ts), or hybrids thereof. The polynucleotides may be, for example, or polynucleotide sequences of DNA or RNA. The DNA or RNA molecules may be, for example, but are not limited to: complementary DNA (cDNA), genomic DNA, synthesized DNA, recombinant DNA, or a hybrid thereof or an RNA molecule such as, for example, mRNA. Accordingly, as used herein, the terms “polynucleotide molecules”, “oligonucleotide”, “polynucleotide”, “nucleic acid” and “nucleotide” sequences are meant to refer to both DNA and RNA molecules. The terms further include oligonucleotides composed of naturally occurring bases, sugars, and covalent inter nucleoside linkages, as well as oligonucleotides having non-naturally occurring portions, which function similarly to respective naturally occurring portions. As used herein, nucleotides (A, G, C or T) and nucleotide sequences are marked in lowercase letters (a, g, c or t)

The terms “polypeptide,” “peptide” and “protein” are used interchangeably herein to refer to a polymer of amino acid residues. The terms also apply to amino acid polymers in which one or more amino acid residue is an artificial chemical analogue of a corresponding naturally occurring amino acid, as well as to naturally occurring amino acid polymers. In some embodiments, one or more of amino acid residue in the polypeptide, can contain modification, such as but be not limited only to, glycosylation, phosphorylation or disulfide bond shape. Also provided are conservative amino acid variants of the peptides and protein molecules disclosed herein. Variants according to the invention also may be made that conserve the overall molecular structure of the encoded proteins or peptides. Given the properties of the individual amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved. As used herein, Amino acids and peptide sequences are marked using conventional Amino Acid nomenclature (single letter or 3-letters code). For example, amino acid “Serine” may be marked as “Ser” or “S” and amino acid “Cysteine” may be marked as “Cys” or “C”.

As referred to herein, the term “complementarity” is directed to base pairing between strands of nucleic acids. As known in the art, each strand of a nucleic acid may be complementary to another strand in that the base pairs between the strands are non-covalently connected via two or three hydrogen bonds. Two nucleotides on opposite complementary nucleic acid strands that are connected by hydrogen bonds are called a base pair. According to the Watson-Crick DNA base pairing, adenine (A or a) forms a base pair with thymine (T or t) and guanine (G or g) with cytosine (C or c). In RNA, thymine is replaced by uracil (U or u). The degree of complementarity between two strands of nucleic acid may vary, according to the number (or percentage) of nucleotides that form base pairs between the strands. For example, “100% complementarity” indicates that all the nucleotides in each strand form base pairs with the complement strand. For example, “95% complementarity” indicates that 95% of the nucleotides in each strand from base pair with the complement strand. The term sufficient complementarity may include any percentage of complementarity from about 30% to about 100%.

The term “construct”, as used herein refers to an artificially assembled or isolated nucleic acid molecule which may be comprises of one or more nucleic acid sequences, wherein the nucleic acid sequences may be coding sequences (that is, sequence which encodes for an end product), regulatory sequences, non-coding sequences, or any combination thereof. The term construct includes, for example, vectors, plasmids but should not be seen as being limited thereto. The term “regulatory sequence” in some embodiments, refers to DNA sequences, which are necessary to affect the expression of coding sequences to which they are operably linked (connected/ligated). The nature of the regulatory sequences differs depending on the host cells. For example, in prokaryotes, regulatory/control sequences may include promoter, ribosomal binding site, and/or terminators. For example, in eukaryotes regulatory/control sequences may include promoters (for example, constitutive of inducible), terminators enhancers, transactivators and/or transcription factors. A regulatory sequence which is “operably linked” to a coding sequence is ligated in such a way that expression of the coding sequence is achieved under suitable conditions. In some embodiments, a “Construct” or a “DNA construct” refer to an artificially assembled or isolated nucleic acid molecule which comprises a coding region of interest and optionally additional regulatory or non-coding sequences.

As used herein, the term “vector” refers to any recombinant polynucleotide construct (such as a DNA construct) that may be used for the purpose of transformation, i.e. the introduction of heterologous DNA into a host cell. One exemplary type of vector is a “plasmid” which refers to a circular double stranded DNA loop into which additional DNA segments can be ligated. Another exemplary type of vector is a viral vector, wherein additional DNA segments can be ligated into the viral genome. Certain vectors are capable of autonomous replication in a host cell into which they are introduced. The term “Expression vector” refers to vectors that have the ability to incorporate and express heterologous nucleic acid fragments (such as DNA) in a foreign cell. In other words, an expression vector comprises nucleic acid sequences/fragments (such as DNA, mRNA), capable of being transcribed or expressed in a target cell. Many viral, prokaryotic and eukaryotic expression vectors are known and/or commercially available. Selection of appropriate expression vectors is within the knowledge of those having skill in the art. The expression vectors can include one or more regulatory sequences.

As used herein, a “primer” defines an oligonucleotide which is capable of annealing to (hybridizing with) a target nucleotide sequence, thereby creating a double stranded region which can serve as an initiation point for DNA synthesis under suitable conditions.

As used herein, the term “transformation” refers to the introduction of foreign DNA into cells. The terms “transformants” or “transformed cells” include the primary transformed cell and cultures derived from that cell regardless to the number of transfers. All progeny may not be precisely identical in DNA content, due to deliberate or inadvertent mutations. Mutant progeny that have the same functionality as screened for in the originally transformed cell are included in the definition of transformants.

As used herein, the terms “introducing” and “transfection” may interchangeably be used and refer to the transfer of molecules, such as, for example, nucleic acids, polynucleotide molecules, vectors, and the like into a target cell(s), and more specifically into the interior of a membrane-enclosed space of a target cell(s). The molecules can be “introduced” into the target cell(s) by any means known to those of skill in the art, for example as taught by Sambrook et al. Molecular Cloning: A Laboratory Manual, Cold Spring Harbor Laboratory Press, New York (2001), the contents of which are incorporated by reference herein. Means of “introducing” molecules into a cell include, for example, but are not limited to: heat shock, calcium phosphate transfection, PEI transfection, electroporation, lipofection, transfection reagent(s), viral-mediated transfer, injection, and the like, or combinations thereof. The transfection of the cell may be performed on any type of cell, of any origin, such as, for example, human cells, animal cells, plant cells, and the like. The cells may be isolated cells, tissue cultured cells, cell lines, cells present within an organism body, and the like.

The terms “upstream” and “downstream”, as used herein refers to a relative position in a nucleotide sequence, such as, for example, a DNA sequence or an RNA sequence. As well known, a nucleotide sequence has a 5′ end and a 3′ end, so called for the carbons on the sugar (deoxyribose or ribose) ring of the nucleotide backbone. Hence, relative to the position on the nucleotide sequence, the term downstream relates to the region towards the 3′ end of the sequence. The term upstream relates to the region towards the 5′ end of the strand.

As used herein, the term “treating” includes, but is not limited to one or more of the following: abrogating, ameliorating, inhibiting, attenuating, blocking, suppressing, reducing, delaying, halting, alleviating or preventing symptoms associated with a condition. Each possibility represents a separate embodiment of the present invention. In some embodiments, the condition is an immune related condition. In some exemplary embodiments, the condition may be selected from, Asthma, Lupus, inflammatory bowel diseases, and the like.

The terms “Semaphorin 3A”, “sema3A”, “Sema3A” and “Sema 3A” may interchangeably be used. Further, it is to be understood that Semaphorin 3A is interchangeable with any alternative name or synonym of this protein known in the art. Typical Semaphorin 3A synonyms include, but are not limited to, collapsin 1, semaphorin III and Sema3A. The terms refer to a protein or polypeptide, primarily to a human protein. The terms further refer to a nucleic acid encoding for the corresponding polypeptide. The amino acid sequences and encoding nucleotide sequences of wild-type Semaphorin 3A are well known in the art. Nucleic acid sequences can be retrieved in public databases like NCBI. In some embodiments, the Homo sapiens Wild type Sema3A accession number gi|100913215|ref]NM_006080.2| corresponds to SEQ ID NO: 1.

The term “wild type Sema3A”, “WT Sema3A”, “naturally occurring Sema3A” and “un-modified Sema3A” may interchangeably be used. The terms refer to the naturally occurring form of Sema3A (i.e., an endogenous, non-mutated Sema3A or full-length Sema3A). In some embodiments, the WT-Sema3A is from a mammalian origin. In some embodiments, the WT-Sema3A is of human origin. In some embodiments, the WT-Sema3A of human origin has an amino acid sequence as denoted by SEQ ID NO: 1. In some embodiments, WT-Semaphorin 3A as used herein is a human Semaphorin 3A having an amino-acid sequence as set forth in SEQ ID NO: 1. The polynucleotide sequence as set forth in SEQ ID NO: 2 corresponds to the cDNA encoding human WT Semaphorin 3A as set forth in SEQ ID NO: 1.

As used herein the terms “modified Sema3A”, “mutated Sema3A”, “non-naturally occurring Sema3A”, “short-Sema3A” and “T-Sema3A” may interchangeably be used. The terms relate to a mutated/modified form of the corresponding wild-type (WT) or natural form of the Sema3A. In some embodiments, the Sema3A is of human origin. In some embodiments, the Sema3A is of mammalian origin. In some embodiments, the modified Sema3A differs from the corresponding wild type Semaphorin 3A by at least one mutation selected from amino acid substitution(s), and/or deletions(s). In particular, the mutated form of the human Semaphorin 3A includes a replacement of the Serine (S) by a Cysteine (C) amino acid at the position that by comparison of homology corresponds to position 257 of the wild type Semaphorin 3A as shown in SEQ ID NO: 1, as well as a C-terminal truncation/deletion of a stretch of at least 50 amino acids, at least 100 amino acids, at least 150 amino acids, at least 200 amino acids, at least 250 amino acids, or at least 254 consecutive amino acids of the WT Semaphorin 3A. Accordingly, in some embodiments, the modified human Sema3A includes an amino acid sequence as denoted by SEQ ID NO. 3. In some embodiments, a modified Sema3A of an origin other than human may include a corresponding point mutation and/or deletion in the respective WT-Sema3A, which are equivalent or homologous to the mutations introduced in the human WT Sema3A.

According to some embodiments, Semaphorin 3A is an isolated Semaphorin 3A. In some embodiments, T-sema3A is an isolated T-sema3A. According to some embodiments, WT-Sema3A and/or the modified Sema3A is a recombinant protein, polypeptide or peptide. As used herein, the term “isolated” means either: 1) separated from at least some of the components with which it is usually associated in nature with respect of the Wild-Type Sema3A; 2) prepared or purified by a process that involves the hand of man (with respect to WT or modified Sema3A); 3) not occurring in nature (with respect of the modified Sema3A).

In some embodiments, there is further provided a nucleic acid molecule encoding a polypeptide comprising an amino acid sequence of a modified Sema3A, wherein the Serine corresponding to position 257 of the wild type Semaphorin 3A (SEQ ID NO: 1) is replaced by Cysteine, and further includes a C-terminal truncation of 254 amino acids of the wild type Semaphorin 3A (SEQ ID NO: 1). In some embodiments, there is further provided a nucleic acid molecule having a nucleotide sequence as denoted by SEQ ID NO: 4, encoding a polypeptide having an amino acid sequence of the modified Sema3A (having an amino acid sequence as denoted by SEQ ID NO: 3).

In some embodiments, the nucleic acid molecule encoding for the modified Sema3A disclosed herein is preferably at least 50% homologous/identical to the nucleic acid sequence as shown in SEQ ID NO: 2. It is understood that such nucleic acid sequences can also include orthologous/homologous/identical (and thus related) sequences. More preferably, the nucleic acid sequence encoding the provided modified Sema3A is at least 52%, 53%, 55%, 60%, 65%, 70%, 75%, 80%, 85%, 90%, 91%, 92%, 93%, 94%, 95%, 96%, 97%, 98%, or 99% homologous/identical to the nucleic acid sequence as shown in SEQ ID NO: 2, wherein the higher values of sequence identity are preferred.

According to some embodiments, the modified Sema3A may further include a protein tag. As used herein, the term “protein tag” refers to a peptide sequence bound to the N-terminus or C-terminus of the protein. According to some embodiments, the protein tag may comprise a glycoprotein. According to some embodiments, the protein tag may be used for separation, purification and/or identification/tracking of the tagged protein. Non-limiting examples of protein tags include: Myc-Tag, Human influenza hemagglutinin (HA), Flag-Tag, His-Tag, Glutathione-S-Transferase (GST) and a combination thereof. Each possibility represents a separate embodiment of the present invention. In some exemplary embodiments, the tag includes a stretch of 6-8 Histidine residues (“His-tag”). In some embodiments, the tag may be a 8×-His tag (SEQ ID NO: 7), located at the C-terminal end of the modified Sema3A. In some exemplary embodiments, a modified Sema3A with a C-terminal His-tag has an amino acid sequence as denoted by SEQ ID NO: 5. In some exemplary embodiments, the nucleic acid molecule encoding the modified Seam 3A with a C-terminal His-tag has a nucleotide sequence as denoted by SEQ ID NO: 6.). In some embodiments, there is provided a nucleic acid molecule having a nucleotide sequence as denoted by SEQ ID NO: 6, encoding a polypeptide having an amino acid sequence of the modified His-Tagged Sema3A (having an amino acid sequence as denoted by SEQ ID NO: 5).

According to some embodiments, the T-Sema3A may include a protein tag upon production, which may be consequently cleaved and/or removed from T-Sema3A prior to incorporation into a composition or prior to being introduced to cells/administered. Cleavage and/or removal of a tag may be performed by any method known in the art, such as, but not limited to, enzymatic and/or chemical cleaving.

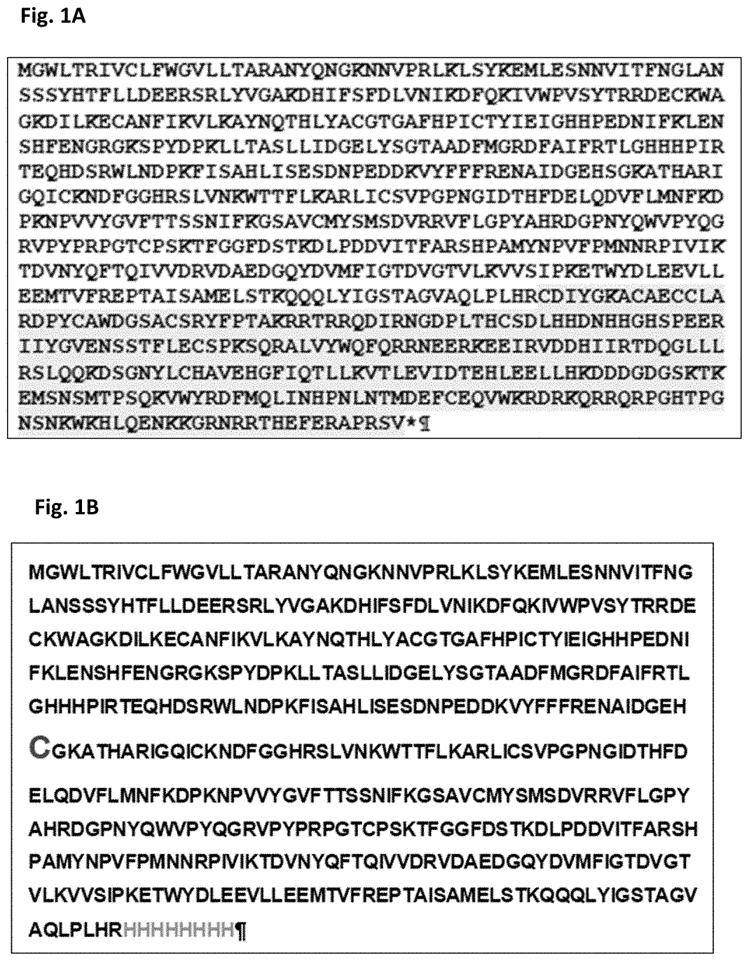

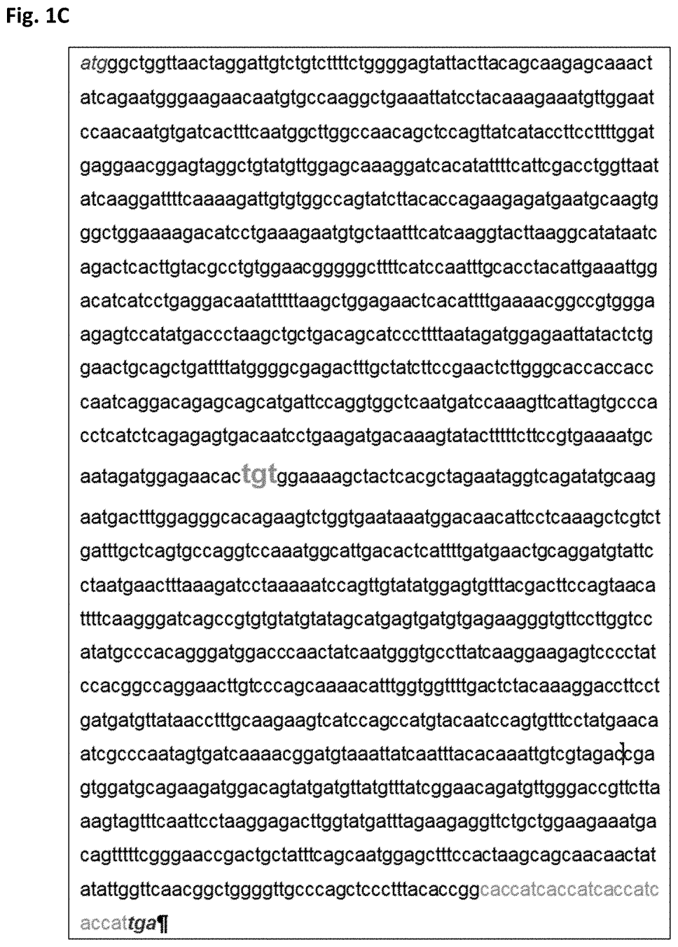

Reference is now made to FIGS. 1A-C, which presents amino acid and/or nucleotide sequences of human WT-Sema3A and modified Sema3A, while highlighting the modifications/differences between the sequences of the WT and modified forms. FIG. 1A presents the 771 amino acid sequence of the human WT-Sema3A (SEQ ID NO: 1), with the C-terminal region (amino acids 517-771, that includes, inter alia, Nrp 1 binding domain and dimerization domain and which is deleted from the corresponding T-Sema3A) marked (gray background). FIG. 1B presents the amino acid sequence of the modified Sema3A, which includes a S257C substitution (marked by Capital C), and a C-terminal truncation at amino acid R516. The modified Sema3A sequence presented in FIG. 1B further includes an in-frame 8×-His tag (HHHHHHHH (SEQ ID NO: 7 (marked)) at the C-terminal end of the modified protein. The amino acid sequence presented in FIG. 1B corresponds to SEQ ID NO: 5. FIG. 1C presents the nucleic acid sequence (cDNA) encoding for the modified tagged Sema3A (SEQ ID NO: 5). The cDNA sequences presented in FIG. 1C corresponds to SEQ ID NO: 6, and includes a codon modification (bases 769-771), whereby the codon encoding for a Serine residue (in the WT Sema3A protein, SEQ ID NO: 2), was changed to a TGT codon encoding cysteine at bases 769-771. In addition, a cDNA sequence encoding 8 histidine residues (caccatcaccatcaccatcaccatcaccat (SEQ ID NO: 8), highlighted) was fused in frame at nucleotide 1548, followed by a stop codon (tga).

According to some embodiments, the modified Sema-3A as disclosed herein may be produced by recombinant or chemical synthetic methods. According to some embodiments, T-Sema3A as disclosed herein may be produced by recombinant methods from genetically-modified host cells. Any host cell known in the art for the production of recombinant proteins may be used for the present invention. According to some embodiments, the host cell is a prokaryotic cell. Representative, non-limiting examples of appropriate prokaryotic hosts include bacterial cells, such as cells of Escherichia coli and Bacillus subtilis. According to other embodiments, the host cell is a eukaryotic cell. According to some exemplary embodiments, the host cell is a fungal cell, such as yeast.

According to some exemplary embodiments, a coding region of interest is a coding region encoding WT-Semaphorin 3A. According to some exemplary embodiments, a coding region of interest is a coding region encoding modified Sema3A. According to some exemplary embodiments, a coding region of interest is a coding region encoding for human modified Sema3A as set forth in SEQ ID NOs: 4 or 6.

In some embodiments, the modified Sema3A may be synthesized by expressing a polynucleotide molecule encoding the modified Sema3A in a host cell, for example, a microorganism cell transformed with the nucleic acid molecule.

In some embodiments, DNA sequences encoding wild type polypeptides, such as Wild-type Semaphorin 3A, may be isolated from any cell producing them, using various methods well known in the art. For example, a DNA encoding the wild-type polypeptide may be amplified from genomic DNA by polymerase chain reaction (PCR) using specific primers, constructed on the basis of the nucleotide sequence of the known wild type sequence. The genomic DNA may be extracted from the cell prior to the amplification using various methods known in the art.

According to some embodiments, the polynucleotide encoding the T-Sema polypeptide may be cloned into any vector known in the art.

According to some embodiments, upon isolation and/or cloning of the polynucleotide encoding the wild type polypeptide, desired mutation(s) may be introduced by modification at one or more base pairs, using methods known in the art, such as for example, site-specific mutagenesis, cassette mutagenesis, recursive ensemble mutagenesis and gene site saturation mutagenesis. Methods are also well known for introducing multiple mutations into a polynucleotide. For example, introduction of two and/or three mutations can be performed using commercially available kits, such as the QuickChange site-directed mutagenesis kit (Stratagene). In some embodiments, as exemplified herein, point mutation is introduced into the sequence encoding for the WT-Semaphorin 3A (represented by SEQ ID NO: 2), whereby nucleotide c—at position 770 (of SEQ ID NO: 2) is replaced/changed to nucleotide g. Such a point mutation results in codon modification (from tct (in the WT) to tgt (in the modified Sema3A) that will translate to a Serine (S or Ser) to Cysteine (C or Cys) amino acid substitution in the peptide expressed therefrom. In addition, the modified Sema3A coding sequence ends at nucleotide 1548 (g) of the corresponding WT-Sema3A (SEQ ID NO: 2). In some embodiments, a stop codon (any Stop codon known in the art, such as, tga, may be placed immediately after (downstream) nucleotide 1548. In some embodiments, a tag may be placed after nucleotide 1548. In some embodiments, a nucleotide sequence encoding for a tag may be placed after nucleotide 1548. In some embodiments, the nucleotide sequence encoding tag may be a His-tag, Myc-tag, FLAG-tag, and the like. In some embodiments, a Stop codon may be placed after the tag-encoding sequence. In some exemplary embodiments, a nucleotide sequence encoding for modified Sema3A, having a stop codon after nucleotide 1548 is represented by SEQ ID NO: 4. For example, a nucleotide sequence encoding for modified Sema3A, having a nucleotide encoding tag (His-tag in this example) followed by a stop codon is represented by SEQ ID NO: 6.

According to some embodiments, an alternative method to producing a polynucleotide with a desired sequence is the use of a synthetic gene. A polynucleotide encoding a desired polypeptide may be prepared synthetically, for example using the phosphoroamidite.

According to some embodiments, the polynucleotide thus produced may then be subjected to further manipulations, including one or more of purification, annealing, ligation, amplification, digestion by restriction endonucleases and cloning into appropriate vectors. The polynucleotide may be ligated either initially into a cloning vector, or directly into an expression vector that is appropriate for its expression in a particular host cell type.

In some embodiments, in case of a fusion protein, or a protein fused with a protein tag, different polynucleotides may be ligated to form one polynucleotide. In some embodiments, the polynucleotide encoding the WT or modified Sema3A polypeptide, may be incorporated into a wide variety of expression vectors, which may be transformed into in a wide variety of host cells.

According to some embodiments, introduction of a polynucleotide into the host cell can be effected by well-known methods, such as chemical transformation (e.g. calcium chloride treatment), electroporation, conjugation, transduction, calcium phosphate transfection, DEAE-dextran mediated transfection, transvection, microinjection, cationic lipid-mediated transfection, scrape loading, ballistic introduction and infection. Representative, non-limiting examples of appropriate hosts include bacterial cells, such as cells of E. coli and Bacillus subtilis.

In some embodiments, the polypeptides may be expressed in any vector suitable for expression. The appropriate vector is determined according to the selected host cell. Vectors for expressing proteins in E. coli, for example, include, but are not limited to, pET, pK233, pT7 and lambda pSKF. Other expression vector systems are based on betagalactosidase (pEX); maltose binding protein (pMAL); and glutathione S-transferase (pGST).

According to some embodiments, as detailed above, the polypeptides may be designed to include a protein tag, for example, a His-Tag (6-8 consecutive histidine residues), which can be isolated and purified by conventional methods.

According to some embodiments, selection of a host cell transformed with the desired vector may be accomplished using standard selection protocols involving growth in a selection medium which is toxic to non-transformed cells. For example, in the case of E. coli, it may be grown in a medium containing an antibiotic selection agent; cells transformed with the expression vector which further provides an antibiotic resistance gene, will grow in the selection medium. In some embodiments, upon transformation of a suitable host cell, and propagation under conditions appropriate for protein expression, the polypeptide may be identified in cell extracts of the transformed cells. Transformed hosts expressing the polypeptide may be identified by analyzing the proteins expressed by the host, for example, using SDS-PAGE and comparing the gel to an SDS-PAGE gel obtained from the host which was transformed with the same vector but not containing a nucleic acid sequence encoding the desired polypeptide.

According to some embodiments, the desired polypeptides which have been identified in cell extracts may be isolated and purified by conventional methods, including ammonium sulfate or ethanol precipitation, acid extraction, salt fractionation, ion exchange chromatography, hydrophobic interaction chromatography, gel permeation chromatography, affinity chromatography, and combinations thereof. The polypeptides of the invention may be produced as fusion proteins, attached to an affinity purification protein tag, such as a His-tag, in order to facilitate their rapid purification.

According to some embodiments, the isolated polypeptide may be analyzed for its various properties, for example, specific activity, using methods known in the art. In a non-limiting example, isolated modified Semaphorin 3A may be analyzed for its ability to bind CD72, lack of binding to Neuropilin 1 receptor, lack of ability to mediate of cell contraction, activation of CD72 signaling (as determined, for example, by increasing phosphorylation of regulatory molecules, such as, STAT-4), inducing/affecting/increasing IL-10 secretion in immune cells (for example, T-cells and/or B-cells), affecting aerobic glycolysis in immune cells (such as, activated T-cells and/or B-cells), and the like, or any combination thereof.

According to some embodiments, a modified Sema3A according to the present invention may also be produced by synthetic means using well known techniques, such as solid phase synthesis. Synthetic polypeptides may be produced using commercially available laboratory peptide design and synthesis kits. In addition, a number of available FMOC peptide synthesis systems are available. Assembly of a polypeptide or fragment can be carried out on a solid support using for example, an Applied Biosystems, Inc. Model 431A automated peptide synthesizer. The polypeptides may be made by either direct synthesis or by synthesis of a series of fragments that can be coupled using other known techniques.

According to some embodiments, there is provided a process for the production of a modified Sema3A polypeptide the process includes culturing/raising a suitable host cells under conditions allowing the expression of the modified Sema3A polypeptide and optionally recovering/isolating the produced polypeptide from the cell culture.

According to some embodiments, there is provided a nucleic acid encoding for the modified Sema3A polypeptide. In some embodiments, there is provide a DNA construct/vector (such as, an expression vector) harboring or comprising a nucleic acid encoding for the modified Sema3A polypeptide (optionally in addition to one or more regulatory sequences, non-coding sequences, and the like).

In some embodiments, various suitable vectors are known to those skilled in art, and the choice of which depends on the function desired. Such vectors include, for example, plasmids, cosmids, viruses, bacteriophages and other vectors. In some embodiments, the polynucleotides and/or vectors harboring the same can be reconstituted into vehicles, such as, for example, liposomes for delivery to target cells. Any cloning vector and/or expression vector known in the art may be used, depending on the purpose, the host cell, and the like. Such vectors may be used for in-vitro and/or in-vivo introduction/expression.

According to some embodiments, the encoding nucleic acid molecules and/or the vectors disclosed herein may be designed for direct introduction or for introduction via carrier, such as, liposomes, viral vectors (adenoviral, retroviral) into target cells.

According to some embodiments, there is provided a host cell harboring or expressing the modified Sema3A. In some embodiments, the host cell may be transformed/transfected with the vector of the present invention or with the nucleic acid encoding for the modified Sema3A. In some embodiments, there is provided a host cell harboring or comprising the nucleic acid molecule of the invention. In some embodiments, the presence of at least one vector or at least one nucleic acid molecule in the host may mediate the expression of the modified Sema3A in the cell. In some embodiments, the nucleic acid molecule or vector comprising the same, may either integrate into the genome of the host cell, or it may be maintained extrachromosomally. In some embodiments, the host cell may be any prokaryotic or eukaryotic cell. In some embodiments, the host cell is a mammalian cell.

According to some embodiments the nucleic acid molecules can be used alone or as part of a vector to express the modified Sema3A polypeptide of the invention in cells, for purification and/or for therapy.

In some embodiments, the nucleic acid molecules (or vectors harboring the same) and/or the modified Sema3A polypeptide, can be used as a medicament (as is, or in the form of a composition, such as a pharmaceutical composition), for treating various conditions, in particular, immune related conditions.

According to some embodiments, there is provided a composition (also referred to herein as pharmaceutical composition) which includes the modified Sema3A polypeptide, the nucleic acid encoding therefor, or vectors harboring the nucleic acids. Each possibility is a separate embodiment. In some embodiments, the composition may include one or more suitable excipients, according to the purpose, type and/or use of the composition. In some embodiments, excipient is a pharmaceutical excipient which may include or a pharmaceutical carrier, vehicle, buffer and/or diluent.

In some embodiments, the composition disclosed herein may be used as a medicament for treating various immune related conditions.

Thus, according to some embodiments, the modified-sema3A (polypeptide or nucleic acid encoding the same) can be used for the successful treatment of various immune-mediated conditions, such as, auto-immune diseases, allergic conditions, conditions related to over activation of the immune system, inflammatory diseases, and the like.

In some embodiments, auto-immune diseases may include such conditions as, but not limited to: Systemic Lupus Erythematosus (SLE), Rheumatoid Arthritis, inflammatory bowel disease (IBD), Uveitis, Psoriasis and the like.

In some embodiments, allergic conditions may include such conditions as, but not limited to: bronchial asthma, allergic conjunctivitis, allergic rhinitis and atopic dermatitis.

In some embodiments, conditions related to over activation of the immune system may include such conditions as, but not limited to: sepsis, cytokine storm-due to infectious diseases and/or inducement by CAR-T, graft-versus host disease (GVHD), and the like.

In some embodiments, inflammatory diseases may include such diseases as, but not limited to: Chronic Obstructive Pulmonary Disease (COPD), Familial Mediterranean fever (FMF), and the like.

According to some embodiments, any suitable route of administration to a subject may be used for the nucleic acid, polypeptide or the composition of the present invention, including but not limited to, local and systemic routes. Exemplary suitable routes of administration include, but are not limited to: orally, intra-nasally, parenterally, intravenously, topically, enema or by inhalation. According to another embodiment, systemic administration of the composition is via an injection. For administration via injection, the composition may be formulated in an aqueous solution, for example in a physiologically compatible buffer including, but not limited, to Hank's solution, Ringer's solution, or physiological salt buffer. Formulations for injection may be presented in unit dosage forms, for example, in ampoules, or in multi-dose containers with, optionally, an added preservative.

According to another embodiment, administration systemically is through a parenteral route. According to some embodiments, parenteral administration is administration intravenously, intra-arterially, intramuscularly, intraperitoneally, intradermally, intravitreally, or subcutaneously. Each of the abovementioned administration routes represents a separate embodiment of the present invention. According to another embodiment, parenteral administration is performed by bolus injection. According to another embodiment, parenteral administration is performed by continuous infusion. According to some embodiments, preparations of the composition of the invention for parenteral administration include sterile aqueous or non-aqueous solutions, suspensions, or emulsions, each representing a separate embodiment of the present invention. Non-limiting examples of non-aqueous solvents or vehicles are propylene glycol, polyethylene glycol, vegetable oils such as olive oil and corn oil, gelatin, and injectable organic esters such as ethyl oleate.

According to another embodiment, parenteral administration is transmucosal administration. According to another embodiment, transmucosal administration is transnasal administration. For transmucosal administration, penetrants appropriate to the barrier to be permeated are used in the formulation. Such penetrants are generally known in the art. The preferred mode of administration will depend upon the particular indication being treated and will be apparent to one of skill in the art.

Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

According to another embodiment, compositions formulated for injection may be in the form of solutions, suspensions, dispersions or emulsions in oily or aqueous vehicles, and may contain formulatory agents such as suspending, stabilizing, and/or dispersing agents. Non-limiting examples of suitable lipophilic solvents or vehicles include fatty oils such as sesame oil, or synthetic fatty acid esters such as ethyl oleate or triglycerides.

According to another embodiment, the composition is administered intravenously, and is thus formulated in a form suitable for intravenous administration. According to another embodiment, the composition is administered intra-arterially, and is thus formulated in a form suitable for intra-arterial administration. According to another embodiment, the composition is administered intramuscularly, and is thus formulated in a form suitable for intramuscular administration.

According to another embodiment, administration systemically is through an enteral route. According to another embodiment, administration through an enteral route is buccal administration. According to another embodiment, administration through an enteral route is oral administration. According to some embodiments, the composition is formulated for oral administration.

According to some embodiments, oral administration is in the form of hard or soft gelatin capsules, pills, capsules, tablets, including coated tablets, dragees, elixirs, suspensions, liquids, gels, slurries, syrups or inhalations and controlled release forms thereof.

According to some embodiments, suitable carriers for oral administration are well known in the art. Compositions for oral use can be made using a solid excipient, optionally grinding the resulting mixture, and processing the mixture of granules, after adding suitable auxiliaries as desired, to obtain tablets or dragee cores. Non-limiting examples of suitable excipients include fillers such as sugars, including lactose, sucrose, mannitol, or sorbitol, cellulose preparations such as, maize starch, wheat starch, rice starch, potato starch, gelatin, gum tragacanth, methyl cellulose, hydroxypropylmethyl-cellulose, and sodium carbomethylcellulose, and/or physiologically acceptable polymers such as polyvinylpyrrolidone (PVP).

In some embodiments, if desired, disintegrating agents, such as cross-linked polyvinyl pyrrolidone, agar, or alginic acid or a salt thereof, such as sodium alginate, may be added. Capsules and cartridges of, for example, gelatin, for use in a dispenser may be formulated containing a powder mix of the composition of the invention and a suitable powder base, such as lactose or starch.

According to some embodiments, solid dosage forms for oral administration include capsules, tablets, pill, powders, and granules. In such solid dosage forms, the composition of the invention is admixed with at least one inert pharmaceutically acceptable carrier such as sucrose, lactose, or starch. Such dosage forms can also comprise, as it normal practice, additional substances other than inert diluents, e.g., lubricating, agents such as magnesium stearate. In the case of capsules, tablets and pills, the dosage forms may also comprise buffering, agents. Tablets and pills can additionally be prepared with enteric coatings.

In some embodiments, liquid dosage forms for oral administration may further contain adjuvants, such as wetting agents, emulsifying and suspending agents, and sweetening, flavoring and perfuming agents. According to some embodiments, enteral coating of the composition is further used for oral or buccal administration. The term “enteral coating”, as used herein, refers to a coating which controls the location of composition absorption within the digestive system. Non-limiting examples for materials used for enteral coating are fatty acids, waxes, plant fibers or plastics.

According to some embodiments, administering is administering topically. According to some embodiments, the composition is formulated for topical administration. The term “topical administration”, as used herein, refers to administration to body surfaces. Non-limiting examples of formulations for topical use include cream, ointment, lotion, gel, foam, suspension, aqueous or cosolvent solutions, salve and sprayable liquid form. Other suitable topical product forms for the compositions of the present invention include, for example, emulsion, mousse, lotion, solution and serum.

According to some embodiments, the administration may include any suitable administration regime, depending, inter alia, on the medical condition, patient characteristics, administration route, and the like. In some embodiments, administration may include administration twice daily, every day, every other day, every third day, every fourth day, every fifth day, once a week, once every second week, once every third week, once every month, and the like.

According to some embodiments, the T-Sema3A polypeptide, the nucleic acid encoding the same, and/or the composition comprising the polypeptide or the nucleic acid molecules, when used for used for treating an immune-related may be used in combination with other therapeutic agents. The components of such combinations may be administered sequentially or simultaneously/concomitantly in separate or combined pharmaceutical formulations by any suitable administration route.

According to some embodiments, there is provided a method of treating an immune related condition, the method includes administration to a subject in need thereof a therapeutically effective amount of modified Sema3A. In some embodiments, the modified Sema3A may be administered as a polypeptide as is, or in a suitable pharmaceutical composition. In some embodiments, the modified Sema3A may be administered as a polynucleotide encoding for the polypeptide as is, or in a suitable pharmaceutical composition.

According to some embodiments, a therapeutically effective amount refers to an amount sufficient to ameliorate and/or prevent at least one of the symptoms associated with an immune-related disorder.

According to some exemplary embodiments, there is provided a method for treating Asthma, the method comprising administering to a subject in need thereof a pharmaceutical composition comprising a therapeutically effective amount of modified Sema3A.

According to some exemplary embodiments, there is provided a method for treating Inflammatory bowel disease, the method comprising administering to a subject in need thereof a pharmaceutical composition comprising a therapeutically effective amount of modified Sema3A.

According to some exemplary embodiments, there is provided a method for treating Lupus, the method comprising administering to a subject in need thereof a pharmaceutical composition comprising a therapeutically effective amount of modified Sema3A.

According to some embodiments, there are provided kits comprising the modified Sema3A peptide and/or the nucleic acid molecule encoding the same and/or the composition as disclosed herein. Such a kit can be used, for example, in the treatment of various immune-related conditions, such as, for example, Asthma, Lupus, and IBD.

In the description and claims of the application, the words “include” and “have”, and forms thereof, are not limited to members in a list with which the words may be associated. As used herein, the term comprising includes the term consisting of.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. According to some embodiments, “about” may specify the value of a parameter to be between 90% and 110% of the given value. According to some embodiments, “about” may specify the value of a parameter to be between 95 % and 105% of the given value.

As used herein, according to some embodiments, the terms “substantially” and “about” may be interchangeable.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Construction of a Modified Sema3A Protein

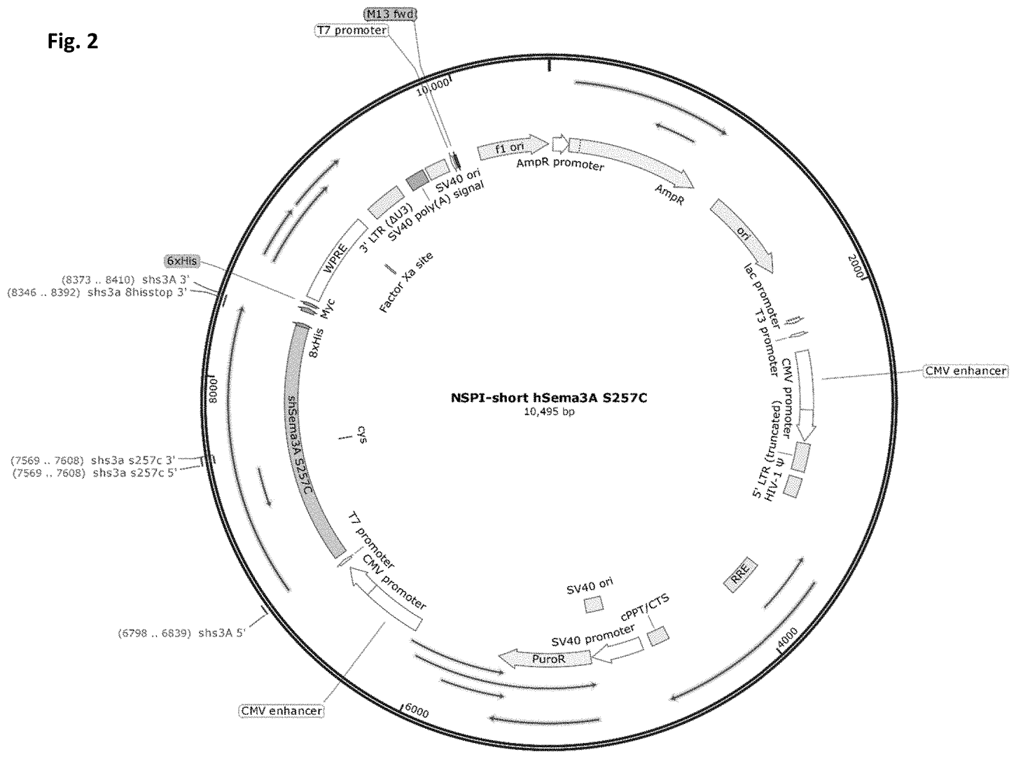

A modified (truncated and mutated) human Sema3A, which retains the signal sequence and the Sema-domain of the WT protein was created. The modified Sema3A (T-Sema3A) was derived from wild type human Semaphorin 3A, using standard genetic engineering techniques. The Sema3A includes a stretch of amino acids 1-516 (compared to the WT Sema3A)) with one point mutation in amino acid 257 (S257C). To this aim, the corresponding region of the Sema3A gene was amplified by PCR using 3 sets of primers (detailed below). The PCR reaction was used to introduce a point mutation at base 770 (from c to g), to result in consequent substitution of amino acid 257 by replacing Serine (in the WT sequence) to Cysteine in the modified Sema3A), in order to allow s-s bonds and the formation of a dimer in the truncated, modified molecule. Additionally, a C-terminal truncation of the sequence was included at nucleotide 1548, to from a truncated modified Sema3A. In some instances, at the 3′ end of the molecule, a nucleotide sequence that is translated to a stretch of 8 Histidine amino acids in included in-frame. The His-tag is followed by a stop codon, thus resulting in the generation of a cDNA encoding the modified Sema3A. The amino acid sequence of such His tagged modified Sema3A is shown in FIG. 1B and is represented by SEQ ID NO: 5. The nucleic acid sequence encoding for such modified Sema3A is shown in FIG. 1C and is represented by SEQ ID NO: 6. In other instances, if a tag sequence is not introduced, an appropriate stop codon is inserted. The amino acid sequence of such modified Sema3A is represented by SEQ ID NO: 3. The nucleic acid sequence encoding for such modified Sema3A is represented by SEQ ID NO: 4. The amplification products were then assembled and ligated into the NSPI-CMV-MCS-myc-His lentiviral expression vector (Shown in FIG. 2), by recombination. This procedure was preformed using NEBuilder HiFi DNA Assembly Master Mix, according to the instructions of the manufacturer (New England Biolabs).

Upon sub-cloning of the modified Sema3A into the NSPI lentiviral expression vector, it was used to infect HEK293 cells (as detailed below). T-sema3A was then purified from the conditioned medium using nickel affinity chromatography.

Primers used in the PCR reaction for the formation of the modified Sema3A, using WT-Sema3A as a template:

shs3A 5′
(SEQ ID NO: 9)
taagcttggtaccgagctcgatgggctggttaactaggattg
shs3a s257c 5′
(SEQ ID NO: 10)
caatagatggagaacactgtggaaaagctactcacgctag
shs3a s257c 3′
(SEQ ID NO: 11)
ctagcgtgagtagcttttccacagtgttctccatctattg
shs3a 8his + stop 3′
(SEQ ID NO: 12)
tcaatggtgatggtgatggtgatggtgccggtgtaaagggagctggg
shs3 A 3′
(SEQ ID NO: 13)
caccacactggactagtgtcaatggtgatggtgatggt

### Example 2—Expression and Purification of Modified Sema3A Polypeptide

The cDNA encoding the modified-sema3A was subcloned into the NSPI lentiviral expression vector, as detailed above. Lentiviruses directing expression of the T-sema3A were generated in HEK293-T cells as previously described (Varshaysky, A., et.al., (2008) Cancer Res. 68, 6922-6931) and used to infect HEK293 cells. Serum free conditioned medium was collected 48 hours after infection and purified on a Nickel-agarose column as per the instructions of the vendor (“Ni-NTA-QUIAGEN”).

The transfected HEK293 cells were grown to 70% confluence and incubated for 48 h in serum free medium. Conditioned medium was collected and then loaded on 1.5 cm diameter column containing 2 ml Ni-NTA agarose at 4° C. (QIAGEN). The beads were washed twice with 10 ml wash buffer (50 mM phosphate buffer pH-8 containing 100 mM NaCl). Then, the beads were eluted five times using 2 ml elution buffer (50 mM phosphate buffer pH-8 containing 100 mM NaCl and 150 mM imidazole). The peptide concentration was determined using Coomassie blue staining by comparison to known concentration of bovine serum albumin fraction V protein (MP Biomedicals™).

The eluate was subsequently dialyzed against PBS and the purified T-sema3A was kept frozen at −80° C.

### Example 3—binding of WT-Sema3A to Nrp1 and CD72 Receptors

### Materials and methods

### Cells

Parental U87MG: Human glioblastoma cell line (ATCC) originally/endogenously expressing Nrp1.
U87MG-ΔNrp1: U87MG knockout for Nrp1 (achieved by CRISPR-Cas9 method).
U87MG-ΔNrp1+CD72: U87MG knockout for Nrp1 and stably express CD72. These cells were generated by introducing full-length CD72 cDNA (Human CD72 9432 bp sequence, clone 5226648, Dharmacon™) into pLenti6.3/V5-DEST lentiviral expression vector in frame with a C-terminal V5 tag (Gateway, Thermo Fisher Scientific). By infection, this vector was introduced to U87MG ΔNrp1, followed by Blasticidin selection.

### Sema3A-Alkaline Phosphatase Concentration

HEK293-Sema3A-AP cells (HEK-293 transfected with WT-Sema3A in frame with alkaline phosphatase) were grown to 70% confluence and incubated for 48 h in serum free medium. Conditioned medium was concentrated using 30 KDa Amicon Ultra centrifugal filter devices for 50-fold concentration.

The Sema3A-AP concentration was determined using Coomassie blue staining by comparison to known concentration of BSA.

### Alkaline Phosphatase Colorimetric Assay

Cells were incubated with concentrated Sema3A-AP for 1.5 h at 4° C., followed by PBS wash and 20 min fixation with 4% paraformaldehyde, and 1 h incubation at 65° C. Next, 5-bromo-4-chloro-3-indolyl phosphate and nitro blue tetrazolium (BCIP/NBT) liquid substrates for AP-enzyme (SIGMA) were added for over-night incubation at 4° C. At the next day, a visible yellow-brown precipitate was microscopely demonstrated in case of AP-Sema3A binding and the mean color intensity per field (1 μm/μm²) was analyzed using Image-Pro software.

### Alkaline Phosphatase Colorimetric Assay: Competitive Inhibition Assay

To analyze the kinetics of sema3A binding to CD72 receptor, Sema4D, which is the known ligand of CD72 was used as competitive inhibitor. The phosphatase colorimetric assay was performed with increasing concentration of recombinant human Sema4D protein (0-50 μg/ml) (abcam) with constant concentration of Sema3A-AP (5 μg/ml). The Graph Pad Prism software was used to draw the kinetics graphs and calculate the binding parameters.

### Results

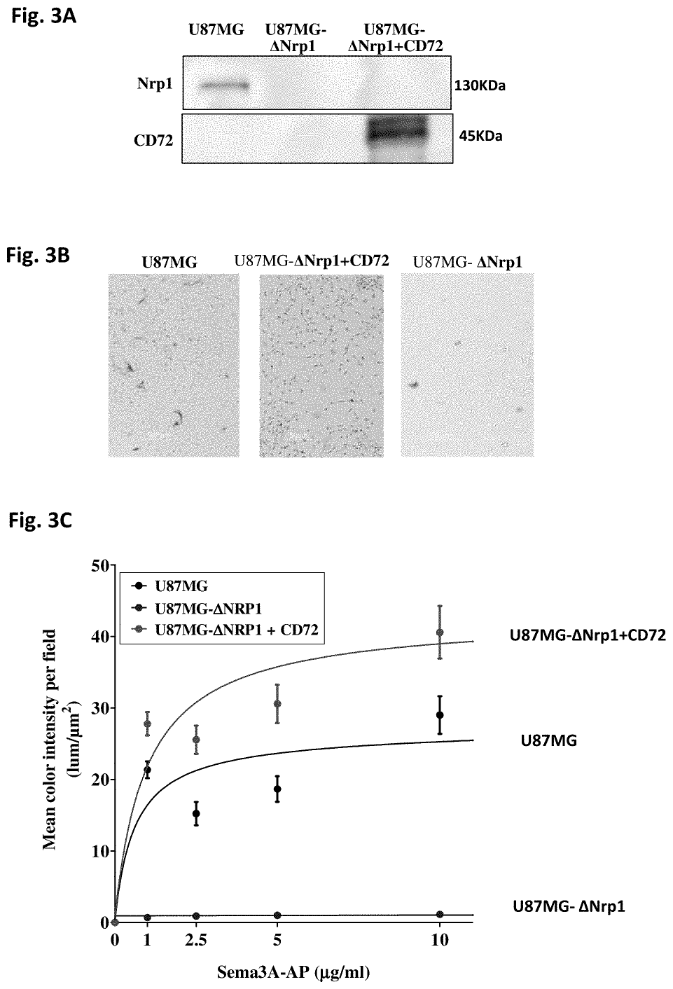

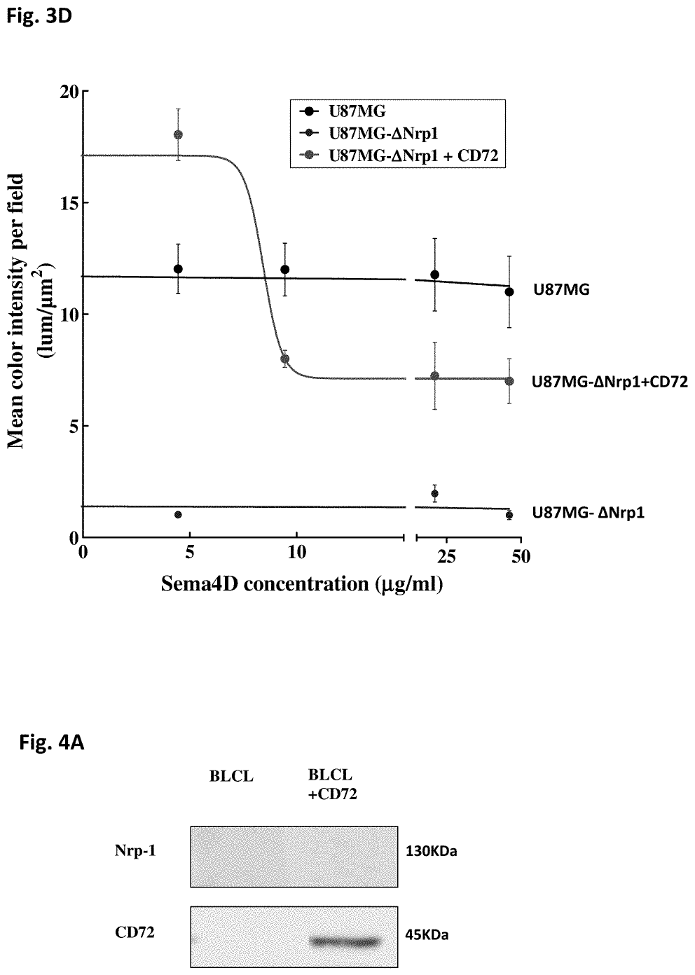

Reference is made to FIG. 3A which shows a pictogram of a Western Blot analysis of modified or non-modified U87MG cells extract, probed with antibodies directed against neuropilin-1 and/or CD72. As can be seen in FIG. 3A, parental U87MG cells (par) express Nrp1, but do not express CD72. U87MG cells in which the gene expressing neuropilin-1 was knocked out using CRISPR/Cas9 (“U87MG-ΔNrp1”) were further infected with empty lentiviruses or lentiviruses directing expression of CD72 (“U87MG-ΔNrp1+CD72”) to which a V5 epitope tag was fused in frame upstream of the stop codon. The results demonstrate that U87MG-ΔNrp1 indeed do not express Nrp1 nor CD72, whereas the U87MG-ΔNrp1+CD72 cells express CD72, and do not express Nrp1.

Next, Sema3A-AP was bound to these cells for 60 minutes at 37° C. The cells were then washed and bound Sema3A-AP was detected using BICP/NBT. The results are presented in the pictogram shown in FIG. 3B.

Next, increasing concentrations of purified Sema3A-AP were incubated for 30 minutes at room temperature with the three cell types. Following incubation (binding), the cells were washed and the amount of bound Sema3A-AP bound per microscopic field assessed using an alkaline phosphatase colorimetric assay. The results are presented in the line graphs of FIG. 3C, which clearly show that WT-Sema3A can bind CD72.

Next, Sema3A-AP (5 μg/ml) was bound/incubated to the three cell types in the presence of increasing concentrations of sema4D, which is an authentic known ligand of CD72. The amount of bound sema3A-AP/microscopic field was then determined. The results presented in FIG. 3D, strengthen the finding that indeed WT-Sema3A can bind CD72 and that the binding affinity of sema3A to CD72 is very similar to its binding affinity to neuropilin-1.

Thus, the results presented in FIGS. 3A-D demonstrate that wild type Sema3A is able to bind the CD72 receptor and that this binding is with similar binding affinity as to Nrp1.

### Example 4—Signal Transduction of Sema3A Using CD72 Receptor

### Materials and methods

### Cells

BLCL (donor #213, healthy female donor): B-Lymphoblastoid Cell Lines, an Epstein-Barr Virus transformed primary B-lymphoblastoid cells (ASTARTE BIOLOGICS, INC.).
BLCL-CD72: BLCL stably express CD72. These cells were generated by introducing full-length CD72 cDNA into pBABE-EGFP lentiviral expression vector (Gateway, Thermo Fisher Scientific). By infection, this vector was introduced to BLCL, followed by EGFP sorting.

### Sema3A Purification

HEK293-Sema3A cells (HEK-293 transfected with Sema3A in frame with a C-terminal His tag) were grown to 70% confluence and incubated for 48 h in serum free medium. Conditioned medium was collected and then loaded on 1.5 cm diameter column containing 2 ml Ni-NTA agarose at 4° C. (QIAGEN). The beads were washed twice with 10 ml wash buffer (50 mM phosphate buffer pH-8 containing 100 mM NaCl). Then, the beads were eluted five times using 2 ml elution buffer (50mM phosphate buffer pH-8 containing 100 mM NaCl and 150 mM imidazole). The Sema3A concentration was determined using Coomassie blue staining by comparison to known concentration of bovine serum albumin fraction V protein (MP Biomedicals™).

### Phosphorylation Assay

Cells were serum starved for 16 h. At the day of the experiment, cells were activated with 5 μg/ml anti IgM for 5 min at 37° C. and then 10 μg/ml of Sema3A or elution buffer as a control were added for extra 10 min at 37° C. The experiment was terminated by a wash with ice cold PBS and lysed with phosphorylation lysis buffer (50 mM Tris-HCl pH-7.5, 150 mM NaCl, 2 mM EDTA, 2 mM EGTA, 5 mM NaF, 2 mM Na₃VO₄, 10 mM Na₄P₂O₇, 1% Triton X-100). 80 μg of proteins were subjected to SDS-PAGE and immunoblotted with an antibody directed against phosphorylated target protein, the blot was then stripped and re-probed with an antibody directed against total protein. Western blots were probed with the following antibodies: anti STAT4 (C-4) (Santa Cruz Biotechnology), anti phospho-STAT4 (Tyr693) (Santa Cruz Biotechnology), p38 MAPK Antibody (Cell Signaling Technology, Inc), Phospho-p38 MAPK (Thr180/Tyr182) Antibody (Cell Signaling Technology, Inc). Quantification of band intensity was performed using ImageQuant LAS 4000 program.

### Results

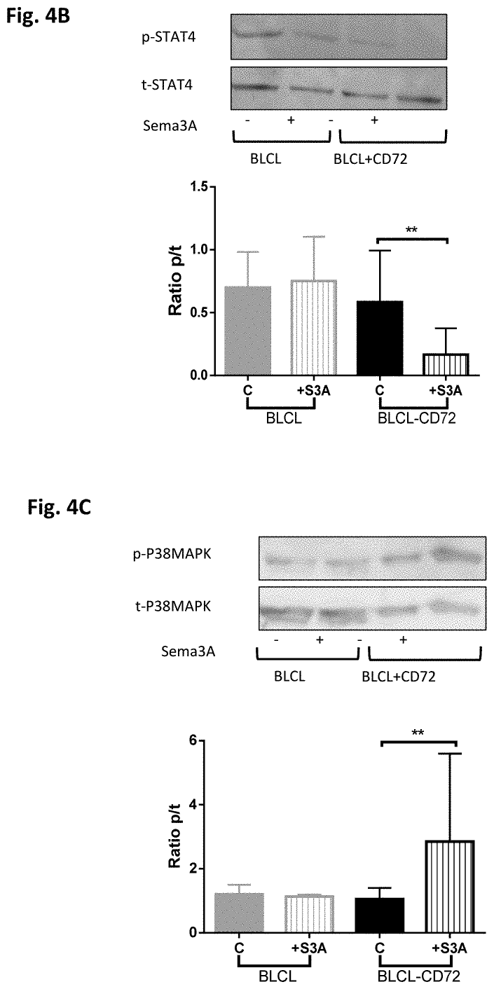

To test whether Sema3A signal transduction is mediated by CD72, Furthermore, CD72 was expressed in primary B-lymphoblastoid cells that lack neuropilin-1. The results presented herein in FIGS. 4A-C demonstrate that Sema3A can signal via CD72 to increase or inhibit the phosphorylation state of several secondary signal transducers in these cells. As shown in FIG. 4A, a primary B-lymphoblastoid cell line (BLCL) was infected with lentiviruses directing expression of CD72. The BLCL cells and the BLCL cells expressing CD72 were probed with antibodies directed against neuropilin-1 (Nrp1) and CD72. parental BLCL cell do not express either of these receptors. Next, BLCL cells and BLCL cells expressing CD72 (BLCL+CD72) were stimulated with WT-Sema3A peptide. The phosphorylation state of Stat-4 and P38 was than determined. The results are shown in FIG. 4B and FIG. 4C which show pictograms of Western Blots of cells extracts probed with antibodies directed against the total proteins and against specific phosphorylation sites in Stat-4 (FIG. 4B) and P38 (FIG. 4C).

Thus, the results demonstrate that Sema3A can bind CD72 and further exert cellular effects via this receptor.

### Example 5—Characterization of the Biological Properties of the Modified-Sema3A

### 1. Endothelial Cells Contraction Assay

HUVECs (Human umbilical vein derived endothelial cells) plated on gelatin plates were incubated with conditioned medium from control HEK2963 cells or with conditioned medium containing similar concentration of wild-type Sema3A or T-Sema3A for 30 minutes (min) in a humidified incubator, at 37° C. After the incubation the cells were photographed using phase-contrast inverted microscope (Ziess).

### 2. CD4+ T Cell Purification and Culturing

Peripheral blood samples from healthy controls were drown to heparin-washed tubes, and then loaded on Lymphoprep—a ficoll gradient to collect PBMCs. CD4+ T cells were positively isolated from PBMCs using anti-human CD4 microbeads (Miltenyi-Biotec) according to the manufacturer's instructions. The purified CD4+ T cells were cultured in plates pre-coated with 10 μg/ml of anti-CD3 for 4 hours at 37° C., then were stimulated with 1 μg/ml of anti-CD28 and 1 μg/ml of IL-2, in addition to purified wild-type Sema3A or T-Sema3A (2-5 μg/ml) for 48 hours at 37° C.

### 3. Flow Cytometry (FACS) Staining

To determine the percentage of T-cells expressing IL-10 after 48 h stimulation with wild-type Sema3A or T-sema3A, CD4+ T cells were stained with FITC-anti-CD4 antibody for 30 min at room temperature, then they were fixed with Fix and Perm medium A for 10 min, afterward they were permeabilized with Fix and Perm medium B, and APC-anti-IL-10 antibody was added for extra 30 min at room temperature. The CD4+ T cells expressing IL-10 were evaluated using Navios EX flow cytometer followed by Kaluza analysis software (Beckman Coulter Life Sciences).

### Results

### Effects on the Cytoskeleton of Endothelial Cells

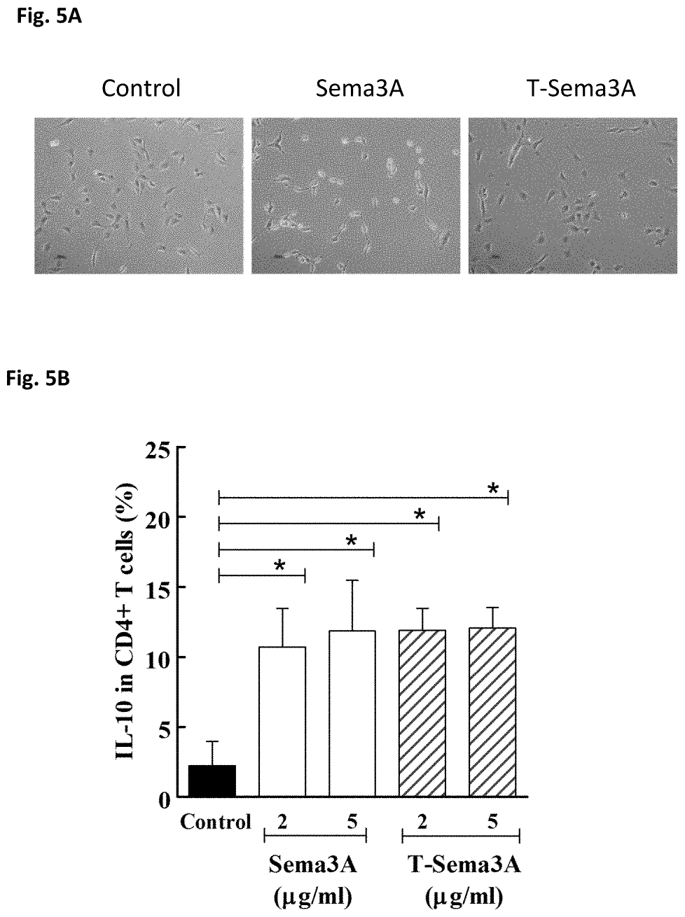

Sema3A binds to the neuropilin-1 receptor which is expressed on endothelial cells. This induces the association of neuropilin-1 with the plexin-A1 and plexin-A4 of the endothelial cells which then transduce a sema3A signal that induces the localized disassembly of the actin cytoskeleton resulting in cell contraction. Thus, Cell contraction in these cells is mediated by the neuropilin-1 receptor. In order to identify whether T-Sema3A has lost its ability to signal using neuropilin-1, cell contraction in human umbilical vein derived endothelial cells (HUVEC) was induced by incubation/stimulation with wild-type Sema3A or T-Sema3A. The results presented in FIG. 5A clearly demonstrate that in contrast to WT Sema3A, T-sema3A failed to induce the contraction of endothelial cells, indicating that it is not able to transduce signals via the neuropilin-1 receptor. Specifically, addition of T-sema3A (0.5-10 μg/ml) to the cells, surprisingly failed to induce the contraction of either human umbilical vein derived endothelial cells, or U87MG glioblastoma cells, whereas wild type Sema3A induced their contraction. The results presented in FIG. 5A clearly show the differential effect of WT-Sema3A and T-Sema3A on the cells contraction. Human umbilical vein derived endothelial cells (HUVEC) were stimulated with conditioned medium from control HEK293 cells (Control), or with conditioned medium containing similar concentrations of Sema3A or T-sema3A derived from HEK293 cells expressing either recombinant Sema3A or T-sema3A. Cells were photographed 30 minutes after addition of the conditioned media.

The results thus implicate that un-like wild-type sema3A, the modified sema3A is unable to induce signal transduction via the neuropilin-1 receptor.

### Effects on CD4+ T Cells

Next, it was sought to identify whether the modified Sema3A can transduce signals via the CD72 receptor. To this aim, increasing concentrations of modified-sema3A or wild type sema3A were added to CD4+ T cells that were activated using anti-CD3 and anti-CD28 for 48 hours. It was found that both short-sema3A and wild type sema3A induced effectively secretion of IL-10, which is the most important anti-inflammatory cytokine secreted by activated CD4+ T cells and T regulatory cells. A concentration of 2 μg\ml was the most effective dose for both wild type sema3A and short-sema3A (FIG. 5B). As shown in FIG. 5B, CD4⁺ T-cells were stimulated with the indicated concentrations of purified Sema3A or T-Sema3A. The percentage of T-cells expressing IL-10 was than determined using FACS analysis.

The results suggest that the modified Sema3A can indeed successfully transduce signals via CD72. The results further suggest that the anti-inflammatory effect of modified-sema3A is at least similar to that of wild type sema3A.

Thus, it can be concluded that modified-sema3A can be used for treatment of immune-related disease, such as, autoimmune diseases, including lupus nephritis or asthma as it would have to be free of side effects associated with the activation of neuropilin-1 mediated signal transduction.

### Example 6: Effect of Modified Sema3A on Metabolic Activity of Activated T-Cells

To determine the effect of modified Sema3A on cellular metabolism (glycolysis) of activated T-cells, the effect on extracellular acidification rate (ECAR) was determined using seahorse technology (Agilent). The aim of the study was to test the ability of T-Sema3A to down regulate aerobic glycolysis in activated immune cells.

As known, the bioenergetic needs of quiescent T cells are met mainly by mitochondrial oxidative phosphorylation (OXPHOS), as a way to generate ATP from a glucose substrate. However, once activated, these cells rapidly proliferate and produce cytokines, therefore, they undergo a metabolic switch, where they utilize aerobic glycolysis as a main source of energy production.

Generally, purified T cells were activated with anti-CD3 and anti-CD28 for 24 hours at 37° C. in the presence or absence of 5 μg of T-Sema3A. At the day of the experiment, cells were harvested and transferred to medium without glucose for 2 hours. The ECAR rate (extracellular acidification rate) was measured based on glycolysis test using the seahorse technology, in accordance with the manufacturer protocol (Seahorse XF technology, Agilent). Briefly, after glucose starvation, glucose is added to the medium. Thereafter, oligomycin is added. Oligomycin, which is an ATP synthase inhibitor, inhibits mitochondrial ATP production, and shifts the energy production to glycolysis, with the subsequent increase in ECAR revealing the cellular maximum glycolytic capacity. Next, 2-deoxy-glucose (2-DG) is added. 2-DG is a glucose analog, which inhibits glycolysis through competitive binding to glucose hexokinase. The resulting decrease in ECAR confirms that the ECAR produced in the experiment is due to glycolysis. The glycolysis phase is measured during the time period between the addition of glucose and the addition of oligomycin. The glycolytic capacity is determined during the time period between the addition of oligomycin and the addition of 2-DG.

More specifically, CD4+ T cells were purified from peripheral blood of healthy controls, according to the manufacturer's instructions (#130-045-101, Miltenyi Biotec) and cultured in plates pre-coated with 10 μg/ml anti-CD3 (#16-0038-85, eBioscience™) for 4 hours at 37° C. and 1 μg/ml anti-CD28 (#16-0289-85, eBioscience™) as activators. In addition, cells were treated with 5 μg/ml T-Sema3A or PBS (as a control) and incubated for 24 hours at 37° C.

On the day of the experiment, cells were harvested and seeded in a 96 well-plate (#102416-100, Seahorse XFe96 FluxPak, Agilent) pre-coated with 22.4 μg/mL cell-tak (#FAL354240, Lapidot Pharma), and incubated in glucose free DMEM basic media supplied with 2 mM Glutamine (ph=7.4) for 2 hours at 37° C.

The sensors cartridge (#102416-100, Seahorse XFe96FluxPak, Agilent) which was hydrated a day earlier was calibrated one hour prior to the experiment and the A, B and C ports were loaded to the final concentrations of 10 mM Glucose, 2 μM Oligomycin and 50 mM 2-DG, respectively. The in live ECAR (extracellular acidification rate) measurements were performed using Agilent Seahorse XF Analyzers. The Glycolysis rate was calculated as (Maximum rate measurement before Oligomycin injection)−(Last rate measurement before Glucose injection). Whereas the Glycolytic Capacity was calculated as (Maximum rate measurement after Oligomycin injection)−(Last rate measurement before Glucose injection) which reflects the maximal rate in which glucose is converted to pyruvate.

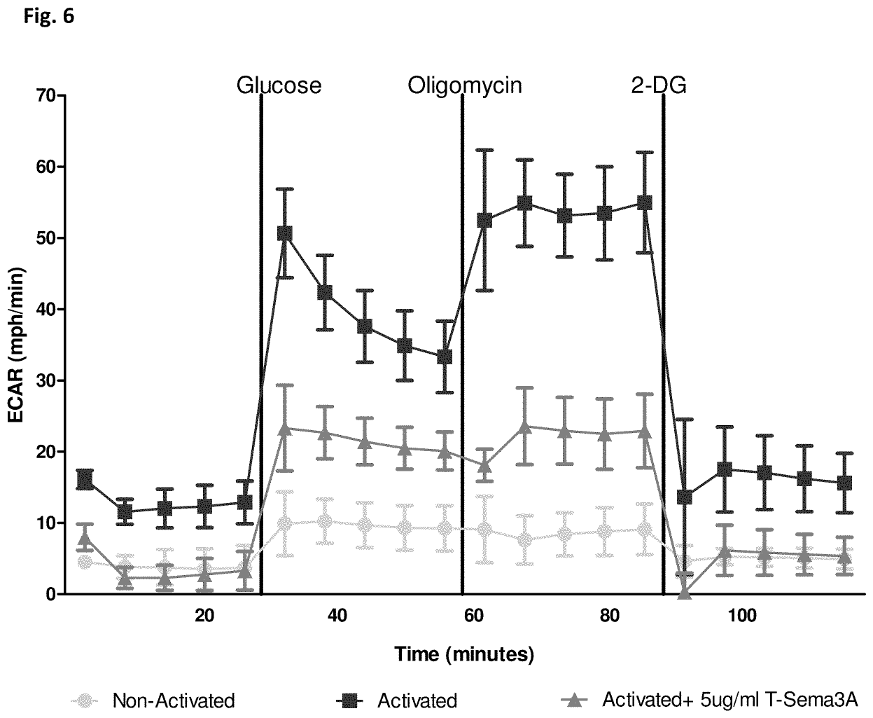

The results are presented in FIG. 6, which clearly shows the effect of T-sema3 on the metabolism of activated T-cells. As can be seen in FIG. 6, the T-sema3A significantly reduces glycolysis and glycolytic capacity in the activated T-cells. The results demonstrate that naïve cells perform minimal glycolysis, whereas activated T cells undergo metabolic switch to aerobic glycolysis. The addition of T-Sema3A to activated T cells decreased the glycolytic rate of these cells, further substantiating the ability of T-Sema3A to down regulate aerobic glycolysis.

Collectively, the results indicate that T-sema3A can reduce metabolism and activity of activated T-cells, further substantiating its effect on the immune system as an immuno-regulator.

### Example 7: In Vivo Studies for Determining Modified Sema3A Effect in Treating Asthma

In order to assess the effect of administration of modified Semaphorin 3A on asthma, the Ovalbumin (OVA)-induced asthma mouse model is utilized. This mouse model is widely used to reproduce the airway eosinophilia, pulmonary inflammation and elevated IgE levels found during asthma. Balb/c female mice are induced for OVA sensitization and airway challenge by intraperitoneal injection with 50 μg ovalbumin (OVA; grade V; Sigma-Aldrich) plus 1 mg Alum hydroxide (Sigma-Aldrich) in 200 μl 0.9% sodium chloride (saline; Hospira) every week and until the end of the experiment. Control group is treated identically except that OVA is absent in the solutions. Modified Sema3A is administered to mice with aerosolized 50 μg recombinant modified Sema3A in 50 μl saline 12 hours prior to each administration of OVA by nasal administration or intraperitoneal administration. Mice are euthanized on day 24 and efficiency of sensitization is assessed as changes in airway function after challenge with aerosolized methacholine (Sigma-Aldrich). The effect of modified Sema3A on airway hyper-responsiveness is compared to the effect of administration of dexamethasone (3 mg\kg), a synthetic member of the glucocorticoid. Mice are anesthetized, tracheostomized, mechanically ventilated, and lung function is assessed starting from 24 h after the final OVA challenge. The lungs are challenged with increasing doses of aerosolized methacholine using flexiVent™ (Scireq—Scientific Respiratory Equipment). Lung resistance is continuously analyzed and compared between the different treatment groups. In addition, serum total IgE levels and assessment of eosinophilia and total inflammatory cells count is assesses on serum samples. The total IgE and OVA-specific IgE levels is measured in serum samples collected from mice on 16 day is determined using enzyme-linked immunosorbent assay (ELISA) kits (Serotec, Oxford, UK) according to the manufacturer's instructions. The absorbance is measured at 450 nm by a micro plate ELISA reader.

Bronchoalveolar lavage fluid (BALF) is taken from the mice and analyzed. BALF is centrifuged, the supernatant is analyzed for inflammatory cell count including eosinophil, lymphocyte, neutrophil, macrophage and total cells, by using direct microscopic counting with a hemocytometer after exclusion of dead cells by trypan blue staining. Th2 cytokines including IL-4 and IL-5 are analyzed in the BALF using an enzyme-linked immunosorbent assay (ELISA) kits (BioSource International, Camarillo, Calif.) according to the manufacturer's protocol.

### Example 8: Examining the Effect of Administration of Modified Sema3A on Systemic Lupus Erythematosus (SLE)

In order to assess how modified Semaphorin 3A affects SLE disease progression in NZB/NZW F1 mice (serving as a model system for SLE), mice are divided into 4 groups:

Prevention Group: In this group, 5 mice are injected with recombinant modified Sema3A on a daily basis and 5 mice are injected with PBS, as a control group. Mice are injected from the age of 6 weeks for 90 days. During this period, both groups are assessed for the development of auto-antibodies (e.g. anti-dsDNA and anti-cardiolipin), kidney function tests (creatinine and BUN), complete blood count on weekly basis and detection of early proteinuria. In addition clinical status of the mice is evaluated by assessing their weight. After this period, the mice are sacrificed and a histological evaluation of their kidneys is performed.

Treatment Group: In this group, 5 mice are injected with recombinant modified Sema3A on a daily basis and 5 mice are injected with PBS, as a control group. Mice are injected from the onset of clinical and laboratory signs of SLE (at four months of age with early proteinuria) and continue for 90 days. During this period, both groups are assessed for the development of auto-antibodies (e.g. anti-dsDNA and anti-cardiolipin), kidney function tests (creatinine and BUN), complete blood count on weekly basis and detection of early proteinuria. In addition clinical status of the mice is evaluated by assessing their weight. After this period, the mice are sacrificed and a histological evaluation of their kidneys is performed.

### Example 9: Examining the Effect of Administration of Modified Sema3A in Inflammatory Bowel Disease (IBD) Model

In the following study, the beneficial effect of T-sema3A in improving the outcome of inflammatory bowel disease (IBD) is tested. I

BD mice model was generated as follow: thirty three 8 week old (W\O) BALB\c female mice were feed with DSS in their water for eight days. Three (3) mice were not treated with DSS and served as naïve group. From the 9^{th} day—the mice were divided into 3 groups: 13 mice were injected intraperitoneal every other day with 50 micrograms of T-Sema3A for 10 days, 13 mice—were injected intraperitoneal every other day with 50 micrograms of control solution and 4 mice—served as disease control, without any treatment. Following 10 days of treatment, mice were sacrificed, their spleen and intestine were removed. T regulatory cells were purified from the spleens and the intestine was subjected to hematoxylin-eosin staining. Serum was also evaluated for pro- and anti-inflammatory cytokines. Consequently, Function of T-regulatory cells is tested, in addition to histopathological results and changes in cytokine status.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. It is to be understood that further trials are being conducted to establish clinical effects.

Listed below are the Amino acid sequences and nucleic acid sequences of wild type or modified Sema3A forms, as disclosed herein.

Wild type Sema3 A polypeptide (Amino Acids)-
SEQ ID NO: 1
MGWLTRIVCLFWGVLLTARANYQNGKNNVPRLKLSYKEMLESNNVITFNGLANSSSYH
TFLLDEERSRLYVGAKDHIFSFDLVNIKDFQKIVWPVSYTRRDECKWAGKDILKECANFI
KVLKAYNQTHLYACGTGAFHPICTYIEIGHHPEDNIFKLENSHFENGRGKSPYDPKLLTA
SLLIDGELYSGTAADFMGRDFAIFRTLGHHHPIRTEQHDSRWLNDPKFISAHLISESDNPE
DDKVYFFFRENAIDGEHSGKATHARIGQICKNDFGGHRSLVNKWTTFLKARLICSVPGP
NGIDTHFDELQDVFLMNFKDPKNPVVYGVFTTSSNIFKGSAVCMYSMSDVRRVFLGPY
AHRDGPNYQWVPYQGRVPYPRPGTCPSKTFGGFDSTKDLPDDVITFARSHPAMYNPVFP
MNNRPIVIKTDVNYQFTQIVVDRVDAEDGQYDVMFIGTDVGTVLKVVSIPKETWYDLE
EVLLEEMTVFREPTAISAMELSTKQQQLYIGSTAGVAQLPLHRCDIYGKACAECCLARD
PYCAWDGSACSRYFPTAKRRTRRQDIRNGDPLTHCSDLHHDNHHGHSPEERIIYGVENS
STFLECSPKSQRALVYWQFQRRNEERKEEIRVDDHIIRTDQGLLLRSLQQKDSGNYLCH
AVEHGFIQTLLKVTLEVIDTEHLEELLHKDDDGDGSKTKEMSNSMTPSQKVWYRDFMQ
LINHPNLNTMDEFCEQVWKRDRKQRRQRPGHTPGNSNKWKHLQENKKGRNRRTHEFE
RAPRSV
Wild type Sema3 A nucleotide sequence (nucleic acids of coding sequence)-
SEQ ID NO: 2
atgggctggt taactaggat tgtctgtctt ttctggggag tattacttac
agcaagagca aactatcaga atgggaagaa caatgtgcca aggctgaaat
tatcctacaa agaaatgttg gaatccaaca atgtgatcac tttcaatggc
ttggccaaca gctccagtta tcataccttc cttttggatg aggaacggag
taggctgtat gttggagcaa aggatcacat attttcattc gacctggtta
atatcaagga ttttcaaaag attgtgtggc cagtatctta caccagaaga
gatgaatgca agtgggctgg aaaagacatc ctgaaagaat gtgctaattt
catcaaggta cttaaggcat ataatcagac tcacttgtac gcctgtggaa
cgggggcttt tcatccaatt tgcacctaca ttgaaattgg acatcatcct
gaggacaata tttttaagct ggagaactca cattttgaaa acggccgtgg
gaagagtcca tatgacccta agctgctgac agcatccctt ttaatagatg
gagaattata ctctggaact gcagctgatt ttatggggcg agactttgct
atcttccgaa ctcttgggca ccaccaccca atcaggacag agcagcatga
ttccaggtgg ctcaatgatc caaagttcat tagtgcccac ctcatctcag
agagtgacaa tcctgaagat gacaaagtat actttttctt ccgtgaaaat
gcaatagatg gagaacactc tggaaaagct actcacgcta gaataggtca
gatatgcaag aatgactttg gagggcacag aagtctggtg aataaatgga
caacattcct caaagctcgt ctgatttgct cagtgccagg tccaaatggc
attgacactc attttgatga actgcaggat gtattcctaa tgaactttaa
agatcctaaa aatccagttg tatatggagt gtttacgact tccagtaaca
ttttcaaggg atcagccgtg tgtatgtata gcatgagtga tgtgagaagg
gtgttccttg gtccatatgc ccacagggat ggacccaact atcaatgggt
gccttatcaa ggaagagtcc cctatccacg gccaggaact tgtcccagca
aaacatttgg tcgttttgac tctacaaagg accttcctga tgatgttata
acctttgcaa gaagtcatcc agccatgtac aatccagtgt ttcctatgaa
caatcgccca atagtgatca aaacggatgt aaattatcaa tttacacaaa
ttgtcgtaga ccgagtggat gcagaagatg gacagtatga tgttatgttt
atcggaacag atgttgggac cgttcttaaa gtagtttcaa ttcctaagga
gacttggtat gatttagaag aggttctgct ggaagaaatg acagtttttc
gggaaccgac tgctatttca gcaatggagc tttccactaa gcagcaacaa
ctatatattg gttcaacggc tggggttgcc cagctccctt tacaccggtg
tgatatttac gggaaagcgt gtgctgagtg ttgcctcgcc cgagaccctt
actgtgcttg ggatggttct gcatgttctc gctattttcc cactgcaaag
agacgcacaa gacgacaaga tataagaaat ggagacccac tgactcactg
ttcagactta caccatgata atcaccatgg ccacagccct gaagagagaa
tcatctatgg tgtagagaat agtagcacat ttttggaatg cagtccgaag
tcgcagagag cgctggtcta ttggcaattc cagaggcgaa atgaagagcg
aaaagaagag atcagagtgg atgatcatat catcaggaca gatcaaggcc
ttctgctacg tagtctacaa cagaaggatt caggcaatta cctctgccat
gcggtggaac atgggttcat acaaactctt cttaaggtaa ccctggaagt
cattgacaca gagcatttgg aagaacttct tcataaagat gatgatggag
atggctctaa gaccaaagaa atgtccaata gcatgacacc tagccagaag
gtctggtaca gagacttcat gcagctcatc aaccacccca atctcaacac
aatggatgag ttctgtgaac aagtttggaa aagggaccga aaacaacgtc
ggcaaaggcc aggacatacc ccagggaaca gtaacaaatg gaagcactta
caagaaaata agaaaggtag aaacaggagg acccacgaat ttgagagggc
acccaggagt gtctga
Modified Sema 3A polypeptide (Amino Acids)-
Seq ID NO: 3
MGWLTRIVCLFWGVLLTARANYQNGKNNVPRLKLSYKEMLESNNVITFNGLANSSSYH
TFLLDEERSRLYVGAKDHIFSFDLVNIKDFQKIVWPVSYTRRDECKWAGKDILKECANFI
KVLKAYNQTHLYACGTGAFHPICTYIEIGHHPEDNIFKLENSHFENGRGKSPYDPKLLTA
SLLIDGELYSGTAADFMGRDFAIFRTLGHHHPIRTEQHDSRWLNDPKFISAHLISESDNPE
DDKVYFFFRENAIDGEHCGKATHARIGQICKNDFGGHRSLVNKWTTFLKARLICSVPGP
NGIDTHFDELQDVFLMNFKDPKNPVVYGVFTTSSNIFKGSAVCMYSMSDVRRVFLGPY
AHRDGPNYQWVPYQGRVPYPRPGTCPSKTFGGFDSTKDLPDDVITFARSHPAMYNPVFP
MNNRPIVIKTDVNYQFTQIVVDRVDAEDGQYDVMFIGTDVGTVLKVVSIPKETWYDLE
EVLLEEMTVFREPTAISAMELSTKQQQLYIGSTAGVAQLPLHR*
Modified Sema3A nucleotide sequence-
SEQ ID NO: 4
atgggctggttaactaggattgtctgtcttttctggggagtattacttacagcaagagcaaact
atcagaatgggaagaacaatgtgccaaggctgaaattatcctacaaagaaatgttggaatccaa
caatgtgatcactttcaatggcttggccaacagctccagttatcataccttccttttggatgag
gaacggagtaggctgtatgttggagcaaaggatcacatattttcattcgacctggttaatatca
aggattttcaaaagattgtgtggccagtatcttacaccagaagagatgaatgcaagtgggctgg
aaaagacatcctgaaagaatgtgctaatttcatcaaggtacttaaggcatataatcagactcac
ttgtacgcctgtggaacgggggcttttcatccaatttgcacctacattgaaattggacatcatc
ctgaggacaatatttttaagctggagaactcacattttgaaaacggccgtgggaagagtccata
tgaccctaagctgctgacagcatcccttttaatagatggagaattatactctggaactgcagct
gattttatggggcgagactttgctatcttccgaactcttgggcaccaccacccaatcaggacag
agcagcatgattccaggtggctcaatgatccaaagttcattagtgcccacctcatctcagagag
tgacaatcctgaagatgacaaagtatactttttcttccgtgaaaatgcaatagatggagaacac
tGtggaaaagctactcacgctagaataggtcagatatgcaagaatgactttggagggcacagaa
gtctggtgaataaatggacaacattcctcaaagctcgtctgatttgctcagtgccaggtccaaa
tggcattgacactcattttgatgaactgcaggatgtattcctaatgaactttaaagatcctaaa
aatccagttgtatatggagtgtttacgacttccagtaacattttcaagggatcagccgtgtgta
tgtatagcatgagtgatgtgagaagggtgttccttggtccatatgcccacagggatggacccaa
ctatcaatgggtgccttatcaaggaagagtcccctatccacggccaggaacttgtcccagcaaa
acatttggtggttttgactctacaaaggaccttcctgatgatgttataacctttgcaagaagtc
atccagccatgtacaatccagtgtttcctatgaacaatcgcccaatagtgatcaaaacggatgt
aaattatcaatttacacaaattgtcgtagaccgagtggatgcagaagatggacagtatgatgtt
atgtttatcggaacagatgttgggaccgttcttaaagtagtttcaattcctaaggagacttggt
atgatttagaagaggttctgctggaagaaatgacagtttttcgggaaccgactgctatttcagc
aatggagctttccactaagcagcaacaactatatattggttcaacggctggggttgcccagctc
cctttacaccggTGA
Modified Sema3A polypeptide with C-terminal His-Tag (Amino Acids)-
Seq ID NO: 5
MGWLTRIVCLFWGVLLTARANYQNGKNNVPRLKLSYKEMLESNNVITFNGLANSSSYH
TFLLDEERSRLYVGAKDHIFSFDLVNIKDFQKIVWPVSYTRRDECKWAGKDILKECANFI
KVLKAYNQTHLYACGTGAFHPICTYIEIGHHPEDNIFKLENSHFENGRGKSPYDPKLLTA
SLLIDGELYSGTAADFMGRDFAIFRTLGHHHPIRTEQHDSRWLNDPKFISAHLISESDNPE
DDKVYFFFRENAIDGEHCGKATHARIGQICKNDFGGHRSLVNKWTTFLKARLICSVPGP
NGIDTHFDELQDVFLMNFKDPKNPVVYGVFTTSSNIFKGSAVCMYSMSDVRRVFLGPY
AHRDGPNYQWVPYQGRVPYPRPGTCPSKTFGGFDSTKDLPDDVITFARSHPAMYNPVFP
MNNRPIVIKTDVNYQFTQIVVDRVDAEDGQYDVMFIGTDVGTVLKVVSIPKETWYDLE
EVLLEEMTVFREPTAISAMELSTKQQQLYIGSTAGVAQLPLHRHHHHHHHH
Modified Sema3A with C-terminal His-Tag nucleotide sequence-
SEQ ID NO: 6
atgggctggttaactaggattgtctgtcttttctggggagtattacttacagcaagagcaaact
atcagaatgggaagaacaatgtgccaaggctgaaattatcctacaaagaaatgttggaatccaa
caatgtgatcactttcaatggcttggccaacagctccagttatcataccttccttttggatgag
gaacggagtaggctgtatgttggagcaaaggatcacatattttcattcgacctggttaatatca
aggattttcaaaagattgtgtggccagtatcttacaccagaagagatgaatgcaagtgggctgg
aaaagacatcctgaaagaatgtgctaatttcatcaaggtacttaaggcatataatcagactcac
ttgtacgcctgtggaacgggggcttttcatccaatttgcacctacattgaaattggacatcatc
ctgaggacaatatttttaagctggagaactcacattttgaaaacggccgtgggaagagtccata
tgaccctaagctgctgacagcatcccttttaatagatggagaattatactctggaactgcagct
gattttatggggcgagactttgctatcttccgaactcttgggcaccaccacccaatcaggacag
agcagcatgattccaggtggctcaatgatccaaagttcattagtgcccacctcatctcagagag
tgacaatcctgaagatgacaaagtatactttttcttccgtgaaaatgcaatagatggagaacac
tGtggaaaagctactcacgctagaataggtcagatatgcaagaatgactttggagggcacagaa
gtctggtgaataaatggacaacattcctcaaagctcgtctgatttgctcagtgccaggtccaaa
tggcattgacactcattttgatgaactgcaggatgtattcctaatgaactttaaagatcctaaa
aatccagttgtatatggagtgtttacgacttccagtaacattttcaagggatcagccgtgtgta
tgtatagcatgagtgatgtgagaagggtgttccttggtccatatgcccacagggatggacccaa
ctatcaatgggtgccttatcaaggaagagtcccctatccacggccaggaacttgtcccagcaaa
acatttggtggttttgactctacaaaggaccttcctgatgatgttataacctttgcaagaagtc
atccagccatgtacaatccagtgtttcctatgaacaatcgcccaatagtgatcaaaacggatgt
aaattatcaatttacacaaattgtcgtagaccgagtggatgcagaagatggacagtatgatgtt
atgtttatcggaacagatgttgggaccgttcttaaagtagtttcaattcctaaggagacttggt
atgatttagaagaggttctgctggaagaaatgacagtttttcgggaaccgactgctatttcagc
aatggagctttccactaagcagcaacaactatatattggttcaacggctggggttgcccagctc
cctttacaccggcaccatcaccatcaccatcaccatcaccatTGA

1.-29. (canceled)

30. A modified Semaphorin 3A polypeptide, said modified Semaphorin 3A polypeptide comprising an amino acid replacement at a position corresponding to position 257 in a wild type Semaphorin 3A protein having an amino acid sequence as denoted by SEQ ID NO: 1, wherein the replacement is with Cysteine (C); and a deletion of at least 100 amino acids of the C-terminal region of the corresponding wild type Semaphorin 3A.

31. The modified Semaphorin 3A polypeptide according to claim 30, wherein the amino acid substitution is S257C and the C-terminal deletion is of amino acids 517-771 of the corresponding wild type Semaphorin 3A.

32. The modified Semaphorin 3A polypeptide according claim 31, wherein the modified Semaphorin 3A and the wild type Semaphorin 3A are of human origin.

33. The modified Semaphorin 3A polypeptide according to claim 30, wherein the polypeptide further comprises a Tag sequence at the N-terminus and/or the C-terminus thereof.

34. The modified Semaphorin 3A polypeptide according to claim 30, comprising an amino acid sequence as denoted by SEQ ID NO: 3.

35. The modified Semaphorin 3A polypeptide according to claim 30, comprising an amino acid sequence as denoted by SEQ ID NO: 5.

36. The modified Semaphorin 3A polypeptide according to claim 30, capable of forming a homo-dimer with a modified Semaphorin 3A polypeptide via S-S bonds formed between Cysteine 257 in each of the modified polypeptides.

37. The modified Semaphorin 3A polypeptide according to claim 30, wherein the polypeptide is capable of binding CD72 receptor.

38. The modified Semaphorin 3A polypeptide according to claim 30, wherein the polypeptide is un-capable of binding to Nrp1 and/or wherein the polypeptide is unable to induce cell contraction.

39. The modified Semaphorin 3A polypeptide according to claim 30, wherein the polypeptide is capable of affecting expression of one or more anti-inflammatory cytokines.

40. The modified Semaphorin 3A polypeptide according to claim 30, capable of inducing expression of IL-10 in CD4+ regulatory T-cells.

41. A composition comprising the modified Semaphorin 3A polypeptide according to claim 30.

42. A nucleic acid molecule encoding the modified Semaphorin 3A of claim 30.

43. The nucleic acid molecule according to claim 42, comprising a nucleotide sequence as denoted by any one of SEQ ID NO: 4 and SEQ ID NO: 6.

44. A vector comprising the nucleic acid molecule of claim 42.

45. A method of treating an immune related condition in a subject in need thereof, the method comprising administering to the subject in need thereof a therapeutically effective amount of the modified Sema3A polypeptide according to claim 30, or a composition comprising the same.

46. A method of treating an immune related disorder in a subject in need thereof, the method comprising administering to the subject in need thereof a therapeutically amount of the nucleic acid molecule according to claim 42, or a vector comprising the same.

47. A host cell comprising the nucleic acid molecule according to claim 42.

48. A host cells transformed or transfected with the vector according to claim 44.

49. A host cell comprising the modified Semaphorin 3A polypeptide according to claim 30.


SEQUENCE LISTING (13 sequences)

SEQ ID NO: 1 - protein, 771 aa, Homo sapiens [sequence omitted]

SEQ ID NO: 2 - DNA, 2316 bp, Homo sapiens
atgggctggttaactaggattgtctgtcttttctggggagtattacttacagcaagagcaaactatcagaatgggaagaacaatgtgccaaggctgaaattatcctacaaagaaatgttggaatccaacaatgtgatcactttcaatggcttggccaacagctccagttatcataccttccttttggatgaggaacggagtaggctgtatgttggagcaaaggatcacatattttcattcgacctggttaatatcaaggattttcaaaagattgtgtggccagtatcttacaccagaagagatgaatgcaagtgggctggaaaagacatcctgaaagaatgtgctaatttcatcaaggtacttaaggcatataatcagactcacttgtacgcctgtggaacgggggcttttcatccaatttgcacctacattgaaattggacatcatcctgaggacaatatttttaagctggagaactcacattttgaaaacggccgtgggaagagtccatatgaccctaagctgctgacagcatcccttttaatagatggagaattatactctggaactgcagctgattttatggggcgagactttgctatcttccgaactcttgggcaccaccacccaatcaggacagagcagcatgattccaggtggctcaatgatccaaagttcattagtgcccacctcatctcagagagtgacaatcctgaagatgacaaagtatactttttcttccgtgaaaatgcaatagatggagaacactctggaaaagctactcacgctagaataggtcagatatgcaagaatgactttggagggcacagaagtctggtgaataaatggacaacattcctcaaagctcgtctgatttgctcagtgccaggtccaaatggcattgacactcattttgatgaactgcaggatgtattcctaatgaactttaaagatcctaaaaatccagttgtatatggagtgtttacgacttccagtaacattttcaagggatcagccgtgtgtatgtatagcatgagtgatgtgagaagggtgttccttggtccatatgcccacagggatggacccaactatcaatgggtgccttatcaaggaagagtcccctatccacggccaggaacttgtcccagcaaaacatttggtggttttgactctacaaaggaccttcctgatgatgttataacctttgcaagaagtcatccagccatgtacaatccagtgtttcctatgaacaatcgcccaatagtgatcaaaacggatgtaaattatcaatttacacaaattgtcgtagaccgagtggatgcagaagatggacagtatgatgttatgtttatcggaacagatgttgggaccgttcttaaagtagtttcaattcctaaggagacttggtatgatttagaagaggttctgctggaagaaatgacagtttttcgggaaccgactgctatttcagcaatggagctttccactaagcagcaacaactatatattggttcaacggctggggttgcccagctccctttacaccggtgtgatatttacgggaaagcgtgtgctgagtgttgcctcgcccgagacccttactgtgcttgggatggttctgcatgttctcgctattttcccactgcaaagagacgcacaagacgacaagatataagaaatggagacccactgactcactgttcagacttacaccatgataatcaccatggccacagccctgaagagagaatcatctatggtgtagagaatagtagcacatttttggaatgcagtccgaagtcgcagagagcgctggtctattggcaattccagaggcgaaatgaagagcgaaaagaagagatcagagtggatgatcatatcatcaggacagatcaaggccttctgctacgtagtctacaacagaaggattcaggcaattacctctgccatgcggtggaacatgggttcatacaaactcttcttaaggtaaccctggaagtcattgacacagagcatttggaagaacttcttcataaagatgatgatggagatggctctaagaccaaagaaatgtccaatagcatgacacctagccagaaggtctggtacagagacttcatgcagctcatcaaccaccccaatctcaacacaatggatgagttctgtgaacaagtttggaaaagggaccgaaaacaacgtcggcaaaggccaggacataccccagggaacagtaacaaatggaagcacttacaagaaaataagaaaggtagaaacaggaggacccacgaatttgagagggcacccaggagtgtctga

SEQ ID NO: 3 - protein, 516 aa, Artificial Sequence
  mutated
MGWLTRIVCLFWGVLLTARANYQNGKNNVPRLKLSYKEMLESNNVITFNGLANSSSYHTFLLDEERSRLYVGAKDHIFSFDLVNIKDFQKIVWPVSYTRRDECKWAGKDILKECANFIKVLKAYNQTHLYACGTGAFHPICTYIEIGHHPEDNIFKLENSHFENGRGKSPYDPKLLTASLLIDGELYSGTAADFMGRDFAIFRTLGHHHPIRTEQHDSRWLNDPKFISAHLISESDNPEDDKVYFFFRENAIDGEHCGKATHARIGQICKNDFGGHRSLVNKWTTFLKARLICSVPGPNGIDTHFDELQDVFLMNFKDPKNPVVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPMNNRPIVIKTDVNYQFTQIVVDRVDAEDGQYDVMFIGTDVGTVLKVVSIPKETWYDLEEVLLEEMTVFREPTAISAMELSTKQQQLYIGSTAGVAQLPLHR

SEQ ID NO: 4 - DNA, 1551 bp, Artificial Sequence
  mutated
atgggctggttaactaggattgtctgtcttttctggggagtattacttacagcaagagcaaactatcagaatgggaagaacaatgtgccaaggctgaaattatcctacaaagaaatgttggaatccaacaatgtgatcactttcaatggcttggccaacagctccagttatcataccttccttttggatgaggaacggagtaggctgtatgttggagcaaaggatcacatattttcattcgacctggttaatatcaaggattttcaaaagattgtgtggccagtatcttacaccagaagagatgaatgcaagtgggctggaaaagacatcctgaaagaatgtgctaatttcatcaaggtacttaaggcatataatcagactcacttgtacgcctgtggaacgggggcttttcatccaatttgcacctacattgaaattggacatcatcctgaggacaatatttttaagctggagaactcacattttgaaaacggccgtgggaagagtccatatgaccctaagctgctgacagcatcccttttaatagatggagaattatactctggaactgcagctgattttatggggcgagactttgctatcttccgaactcttgggcaccaccacccaatcaggacagagcagcatgattccaggtggctcaatgatccaaagttcattagtgcccacctcatctcagagagtgacaatcctgaagatgacaaagtatactttttcttccgtgaaaatgcaatagatggagaacactgtggaaaagctactcacgctagaataggtcagatatgcaagaatgactttggagggcacagaagtctggtgaataaatggacaacattcctcaaagctcgtctgatttgctcagtgccaggtccaaatggcattgacactcattttgatgaactgcaggatgtattcctaatgaactttaaagatcctaaaaatccagttgtatatggagtgtttacgacttccagtaacattttcaagggatcagccgtgtgtatgtatagcatgagtgatgtgagaagggtgttccttggtccatatgcccacagggatggacccaactatcaatgggtgccttatcaaggaagagtcccctatccacggccaggaacttgtcccagcaaaacatttggtggttttgactctacaaaggaccttcctgatgatgttataacctttgcaagaagtcatccagccatgtacaatccagtgtttcctatgaacaatcgcccaatagtgatcaaaacggatgtaaattatcaatttacacaaattgtcgtagaccgagtggatgcagaagatggacagtatgatgttatgtttatcggaacagatgttgggaccgttcttaaagtagtttcaattcctaaggagacttggtatgatttagaagaggttctgctggaagaaatgacagtttttcgggaaccgactgctatttcagcaatggagctttccactaagcagcaacaactatatattggttcaacggctggggttgcccagctccctttacaccggtga

SEQ ID NO: 5 - protein, 524 aa, Artificial Sequence
  mutated
MGWLTRIVCLFWGVLLTARANYQNGKNNVPRLKLSYKEMLESNNVITFNGLANSSSYHTFLLDEERSRLYVGAKDHIFSFDLVNIKDFQKIVWPVSYTRRDECKWAGKDILKECANFIKVLKAYNQTHLYACGTGAFHPICTYIEIGHHPEDNIFKLENSHFENGRGKSPYDPKLLTASLLIDGELYSGTAADFMGRDFAIFRTLGHHHPIRTEQHDSRWLNDPKFISAHLISESDNPEDDKVYFFFRENAIDGEHCGKATHARIGQICKNDFGGHRSLVNKWTTFLKARLICSVPGPNGIDTHFDELQDVFLMNFKDPKNPVVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPMNNRPIVIKTDVNYQFTQIVVDRVDAEDGQYDVMFIGTDVGTVLKVVSIPKETWYDLEEVLLEEMTVFREPTAISAMELSTKQQQLYIGSTAGVAQLPLHRHHHHHHHH

SEQ ID NO: 6 - DNA, 1581 bp, Artificial Sequence
  mutated
atgggctggttaactaggattgtctgtcttttctggggagtattacttacagcaagagcaaactatcagaatgggaagaacaatgtgccaaggctgaaattatcctacaaagaaatgttggaatccaacaatgtgatcactttcaatggcttggccaacagctccagttatcataccttccttttggatgaggaacggagtaggctgtatgttggagcaaaggatcacatattttcattcgacctggttaatatcaaggattttcaaaagattgtgtggccagtatcttacaccagaagagatgaatgcaagtgggctggaaaagacatcctgaaagaatgtgctaatttcatcaaggtacttaaggcatataatcagactcacttgtacgcctgtggaacgggggcttttcatccaatttgcacctacattgaaattggacatcatcctgaggacaatatttttaagctggagaactcacattttgaaaacggccgtgggaagagtccatatgaccctaagctgctgacagcatcccttttaatagatggagaattatactctggaactgcagctgattttatggggcgagactttgctatcttccgaactcttgggcaccaccacccaatcaggacagagcagcatgattccaggtggctcaatgatccaaagttcattagtgcccacctcatctcagagagtgacaatcctgaagatgacaaagtatactttttcttccgtgaaaatgcaatagatggagaacactgtggaaaagctactcacgctagaataggtcagatatgcaagaatgactttggagggcacagaagtctggtgaataaatggacaacattcctcaaagctcgtctgatttgctcagtgccaggtccaaatggcattgacactcattttgatgaactgcaggatgtattcctaatgaactttaaagatcctaaaaatccagttgtatatggagtgtttacgacttccagtaacattttcaagggatcagccgtgtgtatgtatagcatgagtgatgtgagaagggtgttccttggtccatatgcccacagggatggacccaactatcaatgggtgccttatcaaggaagagtcccctatccacggccaggaacttgtcccagcaaaacatttggtggttttgactctacaaaggaccttcctgatgatgttataacctttgcaagaagtcatccagccatgtacaatccagtgtttcctatgaacaatcgcccaatagtgatcaaaacggatgtaaattatcaatttacacaaattgtcgtagaccgagtggatgcagaagatggacagtatgatgttatgtttatcggaacagatgttgggaccgttcttaaagtagtttcaattcctaaggagacttggtatgatttagaagaggttctgctggaagaaatgacagtttttcgggaaccgactgctatttcagcaatggagctttccactaagcagcaacaactatatattggttcaacggctggggttgcccagctccctttacaccggcaccatcaccatcaccatcaccatcaccattga

SEQ ID NO: 7 - protein, 8 aa, Artificial Sequence
  tag
HHHHHHHH

SEQ ID NO: 8 - DNA, 30 bp, Artificial Sequence
  tag
caccatcaccatcaccatcaccatcaccat

SEQ ID NO: 9 - DNA, 42 bp, Artificial Sequence
  Primer
taagcttggtaccgagctcgatgggctggttaactaggattg

SEQ ID NO: 10 - DNA, 40 bp, Artificial Sequence
  Primer
caatagatggagaacactgtggaaaagctactcacgctag

SEQ ID NO: 11 - DNA, 40 bp, Artificial Sequence
  Primer
ctagcgtgagtagcttttccacagtgttctccatctattg

SEQ ID NO: 12 - DNA, 47 bp, Artificial Sequence
  Primer
tcaatggtgatggtgatggtgatggtgccggtgtaaagggagctggg

SEQ ID NO: 13 - DNA, 38 bp, Artificial Sequence
  Primer
caccacactggactagtgtcaatggtgatggtgatggt